(19) United States
(12) Patent Application Publication
(10) Pub. No.: US 2009/0042858 A1
(43) Pub. Date: Feb. 12, 2009

(54) LACTAM CONTAINING HCV INHIBITORS

(76) Inventors: Paul Barsanti, Emeryville, CA (US); Nathan Brammier, Emeryville, CA (US); Bryan Chang, Emeryville, CA (US); Zhi-Jie Ni, Emeryville, CA (US); Weibo Wang, Emeryville, CA (US); Amy Weiner, Emeryville, CA (US)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 11/917,299

(22) PCT Filed: Jun. 16, 2006
(86) PCT No.: PCT/US06/23555

§ 371 Date: Sep. 9, 2008

Related U.S. Application Data
(60) Provisional application No. 60/692,007, filed on Jun. 16, 2005.

(51) Int. Cl. A61K 31/554; C07D 223/12; A61K 31/55; A61P 31/12; C07D 223/16

(52) U.S. Cl. 514/211.06; 540/527; 514/212.03; 540/491

(57) ABSTRACT

The present invention discloses novel methods and compositions for viral inhibition, particularly inhibition of HCV and SARS. The invention also provides compositions including novel oxoazepanylacetamide derivatives useful for viral inhibition.

### FIELD OF INVENTION

The present invention is directed to novel methods and compositions for viral inhibition. In some embodiments, methods are provided for inhibition of HCV and SARS. The invention also is directed to compositions including novel lactam-containing compounds useful for viral inhibition.

### BACKGROUND OF THE INVENTION

Hepatitis is a systemic disease, which predominantly affects the liver. The disease is typified by the initial onset of symptoms such as anorexia, nausea, vomiting, fatigue, malaise, arthralgias, myalgias, and headaches, followed by the onset of jaundice. The disease may also be characterized by increased serum levels of the aminotransferases AST and ALT. Quantification of these enzymes in serum indicates the extent of liver damage.

There are five general categories of viral agents which have been associated with hepatitis: the hepatitis A virus (HAV); the hepatitis B virus (HBV); two types of non-A, non-B (NANB) agents, one blood-borne (hepatitis C) and the other enterically transmitted (hepatitis E); and the HBV-associated delta agent (hepatitis D).

There are two general clinical categories of hepatitis, acute hepatitis and chronic hepatitis. Symptoms for acute hepatitis range from asymptomatic and non-apparent to fatal infections. The disease may be subclinical and persistent, or rapidly progress to chronic liver disease with cirrhosis, and in some cases, to hepatocellular carcinoma. Acute hepatitis B infection in adult Caucasians in the United States progresses to chronic hepatitis B in about 5% to 10% of the cases. In the remainder of the cases, approximately 65% are asymptomatic. In the Far East, infection is usually perinatal, and 50% to 90% progress to the chronic state. It is likely that the different rates of progression are linked to the age at infection rather than genetic differences in the hosts. In the United States, about 0.2% of the population is chronically infected, with higher percentages in high-risk groups such as physicians, drug addicts and renal dialysis patients. In countries and areas such as Taiwan, Hong Kong and Singapore, the level in the population with hepatitis infection may be as high as 10%.

In the United States, about 20% of patients with chronic hepatitis die of liver failure, and a further 5% develop hepatitis B-associated carcinoma. In the Far East, a large percentage of the population is infected with HBV, and after a long chronic infection (20 to 40 years), approximately 25% of these will develop hepatocellular carcinoma.

After the development of serologic tests for both hepatitis A and B, investigators identified other patients with hepatitis-like symptoms, and with incubation periods and modes of transmission consistent with an infectious disease, but without serologic evidence of hepatitis A or B infection. After almost 15 years, the causative agent was identified as an RNA virus. This virus (designated “hepatitis C”) has no homology with HBV, retroviruses, or other hepatitis viruses.

Hepatitis C(HCV) appears to be the major cause of post-transfusion and sporadic non-A, non-B (NANB) hepatitis worldwide, and plays a major role in the development of chronic liver disease, including hepatocellular carcinoma (Kuo et al., Science 244:362-364, 1989; Choo et al., British Medical Bulletin 46(2):423-441, 1990). Of the approximately 3 million persons who receive transfusions each year, approximately 150,000 will develop acute hepatitis C (Davis et al., New Eng. J. Med. 321(22):1501-1506, 1989). In addition, of those that develop acute hepatitis C, at least one-half will develop chronic hepatitis C.

Until recently, no therapy has proven effective for treatment of acute or chronic hepatitis B or C infections, and patients infected with hepatitis must generally allow the disease to run its course. Most anti-viral drugs, such as acyclovir, as well as attempts to bolster the immune system through the use of corticosteroids have proven ineffective (Alter, “Viral hepatitis and liver disease,” Zuckerman (ed.), New York: Alan R— Liss, pp. 53742, 1988). Some anti-viral activity has been observed with adenosine arabinoside (Jacyna et al., British Med. Bull. 46:368-382, 1990), although toxic side effects, which are associated with this drug render such treatment unacceptable.

One treatment that has provided some benefit for chronic hepatitis B and C infections is the use of recombinant alpha interferon (Davis et al., New Eng. J. Med. 321(22):1501-1506, 1989; Perrillo et al., New Eng. J. Med. 323:295-301, 1990). However, for patients with hepatitis B infections only about 35% of infectees responded to such treatment, and in perinatal infectees only about 10% responded to treatment. For hepatitis C infections, despite apparent short-term success utilizing such therapy, six months after termination of treatment half of the patients who responded to therapy had relapsed. In addition, a further difficulty with alpha interferon therapy is that the composition frequently has toxic side effects such as nausea, and flu-like symptoms, which require reduced dosages for sensitive patients.

Hepatocellular carcinoma is a disease that is related to hepatitis B and hepatitis C infections. Briefly, hepatocellular carcinoma is the most common cancer worldwide. It is responsible for approximately 1,000,000 deaths annually, most of them in China and in sub-Saharan Africa. There is strong evidence of an etiologic role for hepatitis B infection in hepatocellular carcinoma. Carriers of the HIBV are at greater than 90 times higher risk for the development of hepatocellular carcinoma than noncarriers. In many cases, hepatitis B virus DNA is integrated within the cellular genome of the tumor. Similarly, hepatitis C virus has also recently been found to be associated with hepatocellular carcinoma, based upon the observation that circulating HCV antibodies can be found in some patients with hepatocellular carcinoma. At present, surgical resection offers the only treatment for hepatocellular carcinoma, as chemotherapy, radiotherapy, and immunotherapy have not shown much promise (Colombo et al., Lancet 1006-1008, 1989; Bisceglie et al., Ann. of Internal Med. 108:390401, 1988; Watanabe et al., Int. J. Cancer 48:340-343, 1991; Bisceglie et al., Amer. J. Gastro. 86:335-338, 1991).

Severe Acute Respiratory Syndrome, or “SARS”, is an often fatal respiratory illness that has recently been reported in Asia, North America, and Europe. The agent responsible for SARS has recently been posited to be a previously unrecognized coronavirus, which has recently been sequenced by the Centers for Disease Control and Prevention (CDC).

Given the severe threat to humans posed by viral infections such as HCV and SARS, it is clear that new therapies for treating such infections are critical importance. This invention is directed to these, as well as other, important ends.

### SUMMARY OF THE INVENTION

In some embodiments, the present invention provides methods for treating a viral infection in a patient suffering therefrom, comprising administering to said patient a therapeutically effective amount of a substituted oxoazepanylacetamide. In some embodiments, the substituted oxoazepanylacetamide is a compound of Formula I:

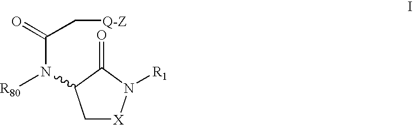

or a stereoisomer or pharmaceutically acceptable salt thereof, wherein:

Q is O, S, SO, SO₂ or N(R₂₅);

R₂₅ is H or alkyl;

R₈₀ is alkyl optionally substituted with up to three independently selected R₆₀ groups, or arylalkyl optionally substituted with up to three independently selected R₃ groups;

each R₃ is independently selected from the group consisting of H, OH, alkyl, alkoxy, alkenyloxy, halogen, aryloxy, heteroaryl, N(R₂₀) (R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl;

wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;

or two R₃ groups, when located on adjacent carbon atoms, together can form a moiety of Formula —(O)ₐ—(CH₂)_{b}—(O)_{c}—(CH₂)_{d}—(O)ₑ— wherein a, c and e are independently 0 or 1, and b and d are independently 0, 1, 2 or 3; provided that said moiety does not contain two adjacent oxygen atoms, and that the sum of a, b, c, d and e is at least 3;

R₁ is selected from the group consisting of H, alkyl, alkenyl, alkynyl, aryl, arylalkyl, heteroaryl and heteroarylalkyl, wherein said alkyl is optionally substituted with up to three independently selected R₆₀ groups, and said alkeny, alkynyl, aryl, arylalkyl heteroaryl and heteroarylalkyl are each optionally substituted with up to three independently selected R₆₁ groups;

each R₆₀ is independently selected from the group consisting of OH, C₁₋₆ alkoxy, C₁₋₆ hydroxyalkyl, C₂₋₆ alkenyl, C₂₋₆ alkynyl, CN, NO₂, —S—C₁₋₆ alkyl, NR₁₂R₁₃, —C(═O)NR₁₂R₁₃, halogen, R₅₀, heteroaryl, heteroarylalkyl, heterocycloalkyl, perhaloalkyl, perhaloalkoxy, amidino, arylalkyloxy, —S-arylalkyl, azido, hydrazino, hydroxylamino, sulfoxy, sulfonyl, sulfide, disulfide, aryl and arylalkyl, wherein said C₁₋₆ alkoxy, C₂₋₆ alkenyl, C₂₋₆ alkynyl, —S—C₁₋₆ alkyl, heteroaryl, heteroarylalkyl, heterocycloalkyl, arylalkyloxy, —S-arylalkyl, aryl and arylalkyl are each optionally substituted with up to three substituents selected from the group consisting of C₁₋₆ alkyl, C₁₋₆ alkoxy, halogen, OH and C₁₋₃ perhaloalkyl;

each R₆₁ is independently selected from the group consisting of R₆₀ and C₁₋₆ alkyl;

X is a single bond, a group of Formula —(CH₂)ₙ— wherein n is 1, 2, 3, 4, or 5; or a group of Formula II:

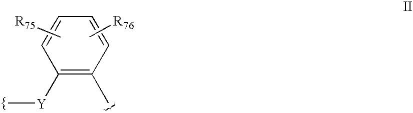

where Y is CH₂, S, SO, SO₂ or N(R₂₀);

R₇₅ and R₇₆ are each independently selected from the group consisting of H, alkyl, alkoxy, alkenyloxy, halogen, aryloxy, heteroaryl, N(R₂₀)(R₂₁) and R₅₀, wherein said alkyloxy, alkenyloxy, aryloxy and heteroaryl are each optionally substituted with up to five independently selected R₆₁ groups, and said alkyl is optionally substituted with up to five independently selected R₆₀ groups;

Z is alkyl, aryl, arylalkyl or heretoaryl, each of which are optionally substituted with up to two independently selected R₂ groups;

each R₂ is independently selected from the group consisting of H, OH, allyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy, arylsulfonyl, and a group of Formula —(CH₂)_{f}—N(R₁₁)—(R₁₀);

wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁, groups; and said alkyl is optionally substituted with up to five independently selected R₆₀ groups;

f is 0, 1, 2, 3, 4, 5 or 6;

R₁₁ is H, alkyl or arylalkyl;

R₁₀ is alkyl, alkenyl, cycloalkyl, aryl, arylalkyl, heteroarylalkyl, or cycloalkylalkyl, wherein said arylalkyl, aryl and heteroaryl are each optionally substituted with are each optionally substituted with up to three independently selected R₆₁ groups; and said alkyl is optionally substituted with up to three independently selected R₆₀ groups;

or R₁₁ and R₁₀ together with the nitrogen to which they are attached can form a heterocyclic ring that is optionally substituted with up to three independently selected R₆₁ groups;

R₁₂ and R₁₃ are each independently H, alkyl or arylalkyl;

R₂₀ and R₂₁ are each independently H, alkyl or arylalkyl, wherein said arylalkyl is optionally substituted with up to three independently selected R₆₁ groups, and said alkyl is optionally substituted with up to three independently selected R₆₀ groups; and

R₅₀ is a group of Formula II:

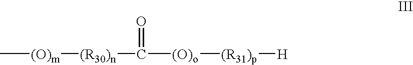

wherein m, n, o and p are each 0 or 1; and
R₃₀ and R₃₁ are each independently C₁₋₆ alkyl.

In some embodiments, the compound, stereoisomer, or pharmaceutically acceptable salt of claim 1 has the Formula IV:

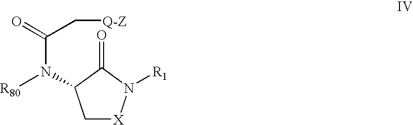

In further embodiments of the compounds of the invention, R₈₀ is benzyl optionally substituted with up to two independently selected R₃ groups; and Z has the Formula V or VI:

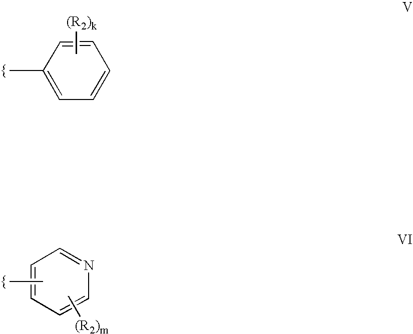

where k and m are each 0, 1 or 2, and each R₂ can be the same or different.

In some further embodiments, each R₃ is independently selected from the group consisting of H, OH, alkyl, alkoxy, alkenyloxy, halogen, aryloxy, heteroaryl, N(R₂₀)(R₂₁), R₅₀, aryl and arylalkyl; wherein said alkoxy, alkenyloxy, aryloxy, heteroaryl, aryl and arylalkyl groups are each optionally substituted with up to five independently selected R₆₁ groups; and said alkyl is optionally substituted with up to five independently selected R₆₀ groups;

or two R₃ groups, when located on adjacent carbon atoms, together can form said moiety of Formula —(O)ₐ—(CH₂)_{b}—(O)_{c}—(CH₂)_{d}—(O)ₑ—; and

each R₂ is independently selected from the group consisting of H, OH, alkyl, alkoxy, alkenyloxy, halogen, aryloxy, heteroaryl, N(R₂₀)(R₂₁), R₅₀, aryl, arylalkyl, and a group of Formula —(CH₂)_{f}—N(R₁₁)—(R₁₀);

wherein said alkoxy, alkenyloxy, aryloxy, heteroaryl, aryl and arylalkyl groups are each optionally substituted with up to five independently selected R₆₁ groups; and said alkyl is optionally substituted with up to five independently selected R₆₀ groups.

In some still further embodiments, R₁ is selected from the group consisting of H, benzyl and alkyl; each R₃ is independently selected from the group consisting of H, OH, C₁₋₆ alkyl, C₁₋₆ alkoxy, CF₃, OCF₃, allyloxy, halogen, pyridyl, —C(═O)—OC₁₋₆ alkyl, thiazolyl optionally substituted with a C₁₋₆ alkyl group, phenoxy optionally substituted with up to three substituents selected from the group consisting of halogen, C₁₋₆ alkoxy, CF₃ and OCF₃; and N(R₄₀)(R₄₁) where R₄₀ is C₁₋₆ alkyl and R₄₁ is C₁₋₆ alkyl that is optionally substituted with —OC₁₋₆ alkyl;

or two R₃ groups, when located on adjacent carbon atoms, together can form said moiety of Formula —(O)ₐ—(CH₂)_{b}—(O)_{c}—(CH₂)_{d}—(O)ₑ—; and

each R₂ is independently selected from the group consisting of H, OH, C₁₋₆ alkyl, C₁₋₆ alkoxy, and a group of Formula —(CH₂)_{f}—N(R₁₁)(R₁₀) wherein: f is 1; R₁₁ is H or C₁₋₆ alkyl; and R₁₀ is an optionally substituted arylalkyl group of Formula —(CH₂)_{g}-L, where g is 0, 1, 2, 3, 4, 5 or 6, and L is selected from the group consisting of H, C₃₋₆ cycloalkyl, allyl, pyridyl and phenyl, wherein said phenyl is optionally substituted with up to three substituents selected from the group consisting of halogen, OH, C₁₋₆ alkyl, C₁₋₆ alkyl, CF₃, OCF₃ and N(R₁₂)(R₁₃);

or R₁₁ and R₁₀ together with the nitrogen to which they are attached can form piperidine that is optionally substituted with a heterocycloalkyl group.

In some further embodiments of each of the foregoing, Z has the Formula V, or Z has the Formula VI. In some further embodiments of each of the foregoing, Q is O, or Q is N(R₂₅), or Q is S, or Q is SO, or Q is SO₂. In still further embodiments of each of the foregoing, X is a group of Formula —(CH₂)ₙ— wherein n is 2 or 3. In further embodiments, X is a group of Formula II wherein Y is CH₂, or Y is S, or Y is SO, or Y is SO₂, or Y is N(R₂₀). In further embodiments of the foregoing, Q is O; Z has the Formula V; and X is a group of Formula —(CH₂)ₙ— wherein n is 2 or 3. In further embodiments of the foregoing, Q is O; Z has the Formula VI; and X is a group of Formula —(CH₂)ₙ— wherein n is 2 or 3.

In further embodiments of the foregoing, Q is S; Z has the Formula V; and X is a group of Formula —(CH₂)ₙ— wherein n is 2 or 3. In further embodiments of the foregoing, Q is S; Z has the Formula VI; and X is a group of Formula —(CH₂)ₙ— wherein n is 2 or 3. In some further embodiments of the foregoing, Q is O; Z has the Formula V; and X is a group of Formula II, wherein Y is CH₂ or S. In some further embodiments of the foregoing, Q is O; Z has the Formula VI; and X is a group of Formula II wherein Y is CH₂ or S. In some further embodiments of the foregoing, Q is S; Z has the Formula V; and X is a group of Formula II, wherein Y is CH₂ or S. In some further embodiments of the foregoing, Q is S; Z has the Formula VI; and X is a group of Formula II, wherein Y is CH₂ or S.

In some further embodiments, compounds of the invention are provided in Table 1, infra.

In some embodiments of the compounds of the invention having Formula I or Formula IV, the compound is not N-(4-ethoxybenzyl)-N-(2-oxoazepan-3-yl)-2-phenoxyacetamide or N-[(2-fluorophenyl)methyl]-N-(2-oxoazepan-3-yl)-2,2-diphenylacetamide.

The present invention further provides methods for alleviating a symptom of a viral infection comprising administering to a patient suffering from said infection a compound of the invention, or a composition comprising a compound of the invention. In some embodiments, the viral infection is HCV.

The present invention further provides methods for alleviating a symptom of SARS comprising administering to a patient suffering therefrom a compound of the invention, or a composition comprising a compound of the invention.

In further embodiments, the present invention provides methods for treating HCV in a patient suffering therefrom, comprising administering to said patient a therapeutically effective amount of a substituted oxoazepanylacetamide, or a substituted oxoazepanylphenoxyacetamide.

In further embodiments, the present invention provides methods for treating SARS in a patient suffering therefrom, comprising administering to said patient a therapeutically effective amount of a substituted oxoazepanylacetamide, or a substituted oxoazepanylphenoxyacetamide.

The present invention further provides methods of inhibiting HCV in a patient comprising administering to said patient a therapeutically effective amount of a compound of the invention.

The present invention further provides methods of inhibiting SARS in a patient comprising administering to said patient a therapeutically effective amount of a compound of the invention.

Also provided in accordance with the present invention are pharmaceutical compositions comprising at least one compound of the invention.

In some embodiments, the present invention provides Compounds of Formula II that display IC50 values of less than 10 μM with respect to inhibition HCV as determined by the assay of Example 83 or Example 84, infra.

The present invention also provides compositions containing the subject compounds, and methods for using the subject compounds. Methodologies for making the compounds of the invention are also disclosed. Other useful methodologies will be apparent to those skilled in the art, once armed with the present disclosure. These and other features of the compounds of the subject invention are set forth in more detail below.

### DETAILED DESCRIPTION

In one aspect, the present invention is directed to novel methods and compositions for inhibition of viral infections, particularly HCV and SARS. In some embodiments, the present invention provides methods for alleviating a symptom of a viral infection, and methods for treating a viral infection, comprising administering to a patient suffering from said infection a compound of the invention. In further embodiments, the invention provides methods for inhibiting HCV or SARS, comprising administering to a patient suffering therefrom a compound of the invention. In some embodiments of the methods of the invention, the compound of the invention is a substituted oxoazepanylacetamide. In further embodiments, the compound is a substituted a substituted oxoazepanylphenoxyacetamide.

In some embodiments, the substituted oxoazepanylacetamide has the Formula I:

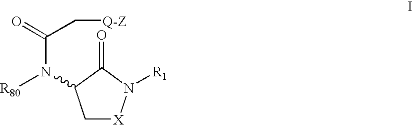

or a stereoisomer or pharmaceutically acceptable salt thereof, wherein:

Q is O, S, SO, SO₂ or N(R₂₅);

R₂₅ is H or alkyl;

R₈₀ is alkyl optionally substituted with up to three independently selected R₆₀ groups, or arylalkyl optionally substituted with up to three independently selected R₃ groups;

each R₃ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl;

wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;

or two R₃ groups, when located on adjacent carbon atoms, together can form a moiety of Formula —(O)ₐ—(CH₂)_{b}—(O)_{c}—(CH₂)_{d}—(O)ₑ— wherein a, c and e are independently 0 or 1, and b and d are independently 0, 1, 2 or 3; provided that said moiety does not contain two adjacent oxygen atoms, and that the sum of a, b, c, d and e is at least 3;

R₁ is selected from the group consisting of H, alkyl, alkenyl, alkynyl, aryl, arylalkyl, heteroaryl and heteroarylalkyl, wherein said alkyl is optionally substituted with up to three independently selected R₆₀ groups, and said alkeny, alkynyl, aryl, arylalkyl heteroaryl and heteroarylalkyl are each optionally substituted with up to free independently selected R₆₁ groups;

each R₆₀ is independently selected from the group consisting of OH, Cab alkoxy, C₁₋₆ hydroxyalkyl, C₂₋₆ alkenyl, C₂₋₆ alkynyl, CN, NO₂, —S—C₁₋₆ alkyl, NR₁₂R₁₃, —C(═O)NR₁₂R₁₃, halogen, R₅₀, heteroaryl, heteroarylalkyl, heterocycloalkyl, perhaloalkyl, perhaloalkoxy, amidino, arylalkyloxy, —S-arylalkyl, azido, hydrazino, hydroxylamino, sulfoxy, sulfonyl, sulfide, disulfide, aryl and arylalkyl, wherein said C₁₋₆ alkoxy, C₂₋₆ alkenyl, C₂₋₆ alkynyl, —S—C₁₋₆ alkyl, heteroaryl, heteroarylalkyl, heterocycloalkyl, arylalkyloxy, —S-arylalkyl, aryl and arylalkyl are each optionally substituted with up to three substituents selected from the group consisting of C₁₋₆ alkyl C₁₋₆ alkoxy, halogen, OH and C₁₋₃ perhaloalkyl;

each R₆₁ is independently selected from the group consisting of R₆₀ and C₁₋₆ alkyl;

X is a single bond, a group of Formula (CH₂)ₙ— wherein n is 1, 2, 3, 4, or 5; or a group of Formula II:

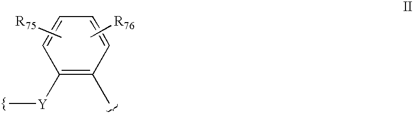

where Y is CH₂, S, SO, SO₂ or N(R₂₀);

R₇₅ and R₇₆ are each independently selected from the group consisting of H, alkyl, alkoxy, alkenyloxy, halogen, aryloxy, heteroaryl, N(R₂₀)(R₂₁) and R₅₀, wherein said alkyloxy, alkenyloxy, aryloxy and heteroaryl are each optionally substituted with up to five independently selected R₆₁ groups, and said alkyl is optionally substituted with up to five independently selected R₆₀ groups;

Z is alkyl, aryl, arylalkyl or heretoaryl, each of which are optionally substituted with up to two independently selected R₂ groups;

each R₂ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoallyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy, arylsulfonyl, and a group of Formula —(CH₂)_{f}—N(R₁₁)—(R₁₀);

wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;

f is 0, 1, 2, 3, 4, 5 or 6;

R₁₁ is H, alkyl or arylalkyl;

R₁₀ is alkyl, alkenyl, cycloalkyl, aryl, arylalkyl, heteroarylalkyl, or cycloalkylalkyl, wherein said arylalkyl, aryl and heteroaryl are each optionally substituted with are each optionally substituted with up to three independently selected R₆₁ groups; and said alkyl is optionally substituted with up to three independently selected R₆₀ groups;

or R₁₁ and R₁₀ together with the nitrogen to which they are attached can form a heterocyclic ring that is optionally substituted with up to three independently selected R₆₁ groups;

R₁₂ and R₁₃ are each independently H, alkyl or arylalkyl;

R₂₀ and R₂₁ are each independently H, alkyl or arylalkyl, wherein said arylalkyl is optionally substituted with up to three independently selected R₆₁ groups, and said alkyl is optionally substituted with up to three independently selected R₆₀ groups; and

R₅₀ is a group of Formula III:

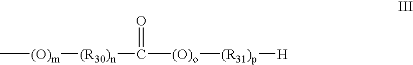

wherein m, n, o and p are each 0 or 1; and
R₃₀ and R₃₁ are each independently C₁₋₆ alkyl.

In some further embodiments, the compounds of the invention have the Formula IV:

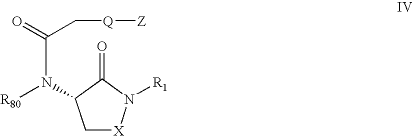

In some further embodiments of the compounds of Formula IV, R₈₀ is benzyl optionally substituted with up to two independently selected R₃ groups; and Z has the Formula V or VI:

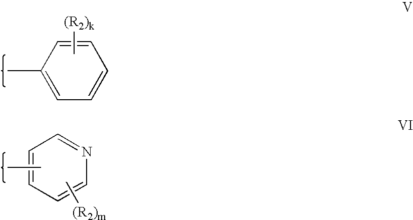

where k and m are each 0, 1 or 2, and each R₂ can be the same or different.

For some embodiments of the invention the term substituted oxoazepanylacetamide refers to a compound having a scaffold of the Formula:

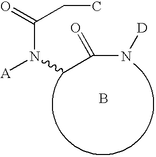

wherein A is an optionally substituted alkyl, aryl or arylalkyl group; ring B is an optionally substituted 5-9 member ring optionally containing an optionally substituted aryl or heteroaryl ring fused thereto; C is a group of Formula -Q-Z as defined supra, and D is a group R₁ as defined supra. In other embodiments of the invention, the term substituted oxoazepanylacetamide refers to scaffolds as described above, having the Formula:

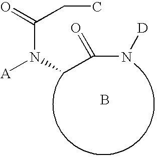

As used herein the term alkyl is intended to mean saturated hydrocarbon species, including straight, branched chain and cyclic hydrocarbons (i.e. “cycloalkyl” groups), for example, methyl, ethyl, n-propyl, isopropyl, n-butyl, sec-butyl, t-butyl, n-pentyl, sec-pentyl, t-pentyl, neopentyl, cyclopropyl, cyclobutyl, cyclopentyl, and cyclohexyl, saturated multiple ring systems such as decahydronaphthalene and adamantane, and the like, including alkyl-substituted derivatives of the foregoing.

A used herein the term alkenyl is intended to denote an alkyl group that contains one or more carbon-carbon double bonds, and is not aromatic. The term alkynyl is intended to denote an alkyl group that contains one or more carbon-carbon triple bonds, and is not aromatic. The term perhaloalkyl is intended to denote an alkyl group in which all hydrogen atoms have been replaced with halogen atoms.

As used herein, the term alkanoyl is intended to denote a group of Formula —C(═O)alkyl.

As used herein, the term alkoxy is intended to denote a moiety of Formula —O-alkyl. The term perhaloalkoxy is intended to denote an alkoxy group in which all hydrogen atoms have been replaced with halogen atoms. The term “alkoxyalkyl” is intended to denote a group of Formula -alkyl-O-alkyl. The terms monoalkylamino and dialkylamino denote, respectively, groups of Formula —NH-alkyl and N(alkyl)₂, where the constituent alkyl groups can be the same or different. The term “alkylaminoalkyl is intended to denote a group of Formula -alkyl-NR′R″ where R′ is alkyl, and R″ is H (i.e., “monoalkylaminoalkyl”) or alkyl (i.e., dialkylaminoalkyl). The term “alkoxyalkylaminoalkyl” denotes an alkylaminoalkyl group wherein one or both of the R′ and R″ alkyl groups are substituted with an alkoxy group.

As used herein the term aryl is intended to mean an aromatic hydrocarbon system for example phenyl, naphthyl, phenanthrenyl, anthracenyl, pyrenyl, and the like. T some embodiments, aryl groups have from 6 to 10 carbon atoms.

The term “arylalkoxy” is intended to mean an alkoxy group that bears an aryl group. The term “aryloxyalkyl” is intended to denote a group of Formula -alkyl-O-aryl. The term arylcarbonyl is intended to denote a moiety of Formula —C(═O)aryl. The term arylalkanoylalkyl is intended to denote a moiety of Formula alkyl-C(═O)-arylalkyl. The term arylalkyloxy denotes a group of Formula —O-arylalkyl, for example a benzyloxy group. The term alkylheteroaryl denotes a group of Formula -heteroaryl-alkyl, for example a 4-methyl-pyrid-2-yl group.

As used herein, the term arylalkyl (or “aralkyl”) is intended to mean an alkyl group that has an aryl group appended thereto, for example benzyl and naphthylmethyl groups. In some embodiments, arylalkyl groups have from 7 to 11 carbon atoms.

As used herein, the term alkylaryl (or “alkaryl”) is intended to mean an aryl group that has one or more alkyl groups appended thereto, for example a 4-methylphen-1-yl group, or a xylyl group attached through the phenyl ring thereof.

The terms “arylamino”, “arylalkylamino” and “alkarylamino” respectively denote an aryl, arylalkyl or alkylaryl group that is attached through an amino group of Formula —NR″, wherein R″ is H or alkyl. The terms “arylakylaminoalkyl” and “alkylarylaminoalkyl” denote an alkyl group that bears, respectively, an arylalkylamino group or an alkylarylamino group.

As used herein, the term “heterocycloalkyl” is intended to mean a group that contains a nonaromatic ring which contains one or more ring hetero (i.e., non-carbon) atoms which are preferably O, N or S, and which can also contain one or more appended alkyl groups. Also included in the definition of heterocycloalkyl are moieties that contain exocyclic heteroatoms, for example a cycloalkyl ring having a ring carbon attached to an exocyclic O or S atom through a double bond. Also included in the definition of heterocycloalkyl are moieties that having one or more aromatic rings fused (i.e., having a bond in common with) to the nonaromatic heterocyclic ring, for example phthalimidyl, naphthalimidyl pyromellitic diimidyl, phthalanyl, and benzo derivatives of saturated heterocycles such as indolene and isoindolene groups.

The term “heterocycloalkylamino” denotes a heterocycloalkyl group that is attached through an amino group of Formula —NR″, wherein R″ is H or alkyl. The term “heterocycloalkylaminoalkyl” denotes a heterocycloalkylamino group that is attached through an alkyl group. The term “heterocycloalkylally” denotes a heterocycloalkyl group that is attached through an exocyclic alkyl group thereof. The term “heterocycloalkylalkylaminoalkyl” denotes a group of Formula -alkyl-NR″-heterocycloalkylalkyl, wherein R″ is H or alkyl.

As used herein, the term “heteroaryl” means an aryl group that contains one or more ring hetero (i.e., non-arbon) atoms, which are preferably O, N or S. In some embodiments, heteroaryl groups are monocyclic or bicyclic, and have up to four ring hetero atoms. Examples of some preferred heteroaryl groups include radicals derived from pyrrole, pyrazole, imidazole, triazoles, tetrazole, pyridine, pyrazine, pyridazine, pyrimidine, triazines, quinolines, indoles, benzimidazoles, and the like.

The term “heteroarylcarbonyl” is intended to denote a moiety of Formula —C(═O)-heteroaryl. The term “heteroarylalkyl” is intended to denote a group of Formula -alkyl-heteroaryl. The term “alkylheteroaryl” is intended to denote a group of Formula -heteroaryl-alkyl. The term “heteroarylalkylamino” denotes a group of Formula —NR″-heteroarylalkyl, wherein R″ is H or alkyl. The term “heteroarylalkylaminoalkyl” denotes a group of Formula -alkyl-heteroarylalkylamino.

The term “halogen” is intended to denote a Group VII element, including include fluorine, chlorine, bromine and iodine.

In general, the suffix “sulfonyl” is intended to mean attachment of the group through a group having the Formula —S(═O)₂—. Thus, the term “alkylsulfonyl” is intended to denote a group of Formula —SO₂-alkyl, the term arylsulfonyl is intended to mean a moiety of Formula —S(═O)₂-aryl, and the term heteroarylsulfonyl is intended to mean a moiety of Formula —S(═O)₂-heteroaryl.

In general, a term containing the suffix “oxy” is intended to mean attachment of the group through an oxygen atom. For example, the term “aryloxy” is intended to mean an aryl group attached through an oxygen atom, for example phenoxy, and the term “aryalkyloxy” or “arylalkyloxy” denotes a group of Formula —O-arylalkyl which is equivalent to aryl-alkyl-O— which is also equivalent to —O-alkyl-aryl.

As used herein, the term aryloxycarbonyl is intended to men a moiety of Formula —C(═O)—O-aryl, for example phenoxycarbonyl.

As used herein, the term alkoxyalkoxyalkyl is intended to mean a moiety of Formula -alkyl-O-alkyl-O-alkyl.

As used herein, the term hydroxyalkyl is intended to mean an alkyl group that has a hydrogen atom thereof replaced with OH.

As used herein, the term alkoxycarbonyl is intended to mean a moiety of Formula —C(═O)—O-alkyl.

The term “side chain of a naturally occurring alpha amino acid” is intended to mean the side chain of naturally occurring alpha amino acids, with the exception of glycine, that are known to have the Formula H₂N—CHR—COOH, where R is the side chain. Examples of such naturally occurring amino acids include the 20 so called “essential” amino acids, for example serine and threonine. Further side chains of naturally occurring alpha amino acids can be found in Biochemistry, 3rd Edition, Matthews, Van Holde, and Ahern, Addison Wesley Longman, San Francisco, Calif., incorporated by reference herein in its entirety.

In some embodiments, the present invention provides compounds having the Formula IV:

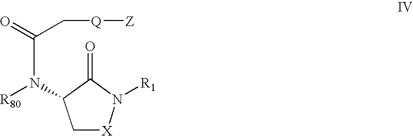

wherein:

R₈₀ is benzyl optionally substituted with up to two independently selected R₃ groups; and

Z has the Formula V or VI:

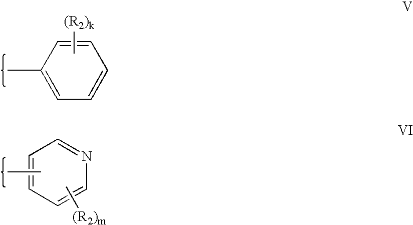

where k and m are each 0, 1 or 2, and each R₂ can be the same or different

In some embodiments of the compounds of the invention, each R₃ is independently selected from the group consisting of H, OH, alkyl, alkoxy, alkenyloxy, halogen, aryloxy, heteroaryl, N(R₂₀)(R₂₁), R₅₀, aryl and arylalkyl; wherein said alkoxy, alkenyloxy, aryloxy, heteroaryl, aryl and arylalkyl groups are each optionally substituted with up to five independently selected R₆₁ groups; and said alkyl is optionally substituted with up to five independently selected R₆₀ groups;

or two R₃ groups, when located on adjacent carbon atoms, together can form said moiety of Formula —(O)ₐ—(CH₂)_{b}—(O)_{c}—(CH₂)_{d}—(O)ₑ—; and

each R₂ is independently selected from the group consisting of H, OH, alkyl, alkoxy, alkenyloxy, halogen, aryloxy, heteroaryl, N(R₂₀)(R₂₁), R₅₀, aryl, arylalkyl, and a group of Formula —(CH₂)_{f}—N(R₁₁)—(R₁₀);

wherein said alkoxy, alkenyloxy, aryloxy, heteroaryl, aryl and arylalkyl groups are each optionally substituted with up to five independently selected R₆₁ groups; and said alkyl is optionally substituted with up to five independently selected R₆₀ groups.

In some still further embodiments, R₁ is selected from the group consisting of H, benzyl and alkyl; each R₃ is independently selected from the group consisting of H, OH, C₁₋₆ alkyl, C₁₋₆ alkoxy, CF₃, OCF₃, allyloxy, halogen, pyridyl, —C(═O)—OC₁₋₆ alkyl, thiazolyl optionally substituted with a C₁₋₆ alkyl group, phenoxy optionally substituted with up to three substituents selected from the group consisting of halogen, C₁₋₆ alkoxy, CF₃ and OCF₃; and N(R₄₀)(R₄₁) where R₄₀ is C₁₋₆ alkyl and R₄₁ is C₁₋₆ alkyl that is optionally substituted with —OC₁₋₆ alkyl;

or two R₃ groups, when located on adjacent carbon atoms, together can form said moiety of Formula —(O)ₐ—(CH₂)_{b}—(O)_{c}—(CH₂)_{d}—(O)ₑ—; and

each R₂ is independently selected from the group consisting of H, OH, C₁₋₆alkyl, C₁₋₆ alkoxy, and a group of Formula —(CH₂)_{f}—N(R₁₁)(R₁₀) wherein: f is 1; R₁₁ is H or C₁₋₆ alkyl; and R₁₀ is a group of Formula —(CH₂)_{g}-L, where g is 0, 1, 2, 3, 4, 5 or 6, and L is selected from the group consisting of H, C₃₋₆ cycloalkyl, allyl, pyridyl and phenyl, wherein said phenyl is optionally substituted with up to three substituents selected from the group consisting of halogen, OH, C₁₋₆ alkyl, OC₁₋₆ alkyl, CF₃, OCF₃ and N(R₁₂)(R₁₃);

or R₁₁ and R₁₀ together with the nitrogen to which they are attached can form piperidine that is optionally substituted with a heterocycloalkyl group.

In some embodiments of the compounds of the invention, Z has the Formula V, or Z has the Formula VI. In some further embodiments, Q is O, or Q is N(R₂₅), or Q is S, or Q is SO, or Q is SO₂. In still further embodiments, X is a group of Formula —(CH₂)ₙ— wherein n is 2 or 3. In further embodiments, X is a group of Formula II wherein Y is CH₂, or Y is S, or Y is SO, or Y is SO₂, or Y is N(R₂₀). In further embodiments, Q is O; Z has the Formula V; and X is a group of Formula —(CH₂)ₙ— wherein n is 2 or 3. In further embodiments, Q is O; Z has the Formula VI; and X is a group of Formula —(CH₂)ₙ— wherein n is 2 or 3.

In further embodiments of the compounds of the invention, Q is S; Z has the Formula V; and X is a group of Formula —(CH₂)ₙ— wherein n is 2 or 3. In further embodiments, Q is S; Z has the Formula VI; and X is a group of Formula —(CH₂)ₙ— wherein n is 2 or 3. In some further embodiments, Q is O; Z has the Formula V; and X is a group of Formula II, wherein Y is CH₂ or S. In some further embodiments, Q is O; Z has the Formula VI; and X is a group of Formula II wherein Y is CH₂ or S. In some further embodiments, Q is S; Z has the Formula V; and X is a group of Formula II, wherein Y is CH₂ or S. In some further embodiments, Q is S; Z has the Formula VI; and X is a group of Formula II, wherein Y is CH₂ or S.

In some further embodiments, compounds of the invention are provided in Table 1, infra.

The substituted oxoazepanylacetamide compounds described herein can be readily synthesized as shown in Scheme 1, the specifics of which are provided in the Examples section.

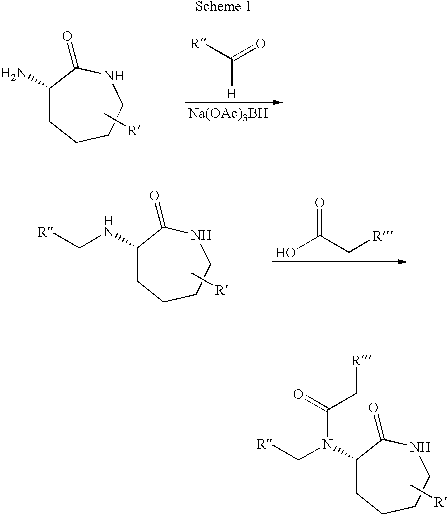

It will be appreciated that by selection of appropriately substituted amino lactam and aldehyde starting materials, a wide variety of substituted oxoazepanylacetamide compounds can be prepared, including those of Formulas (I) and (IV). Thus, in some embodiment, the invention provides for methods of making compounds of Formulas (I) and (IV) according to Scheme 1. It is further contemplated that the instant invention covers the intermediates as well as their corresponding methods of synthesis as described in Scheme 1 and the Examples described below. In accordance with such methods, the constituent variables of the compounds can include any of those same values described for the compounds of Formula (I) and (IV).

It is contemplated that the present invention include all possible protonated and unprotonated forms of the compounds described herein, as well as solvates and pharmaceutically acceptable salts thereof. It also is intended that each of the compounds described herein specifically include all possible tautomers and stereoisomers.

Throughout the present disclosure, compounds are described by generic and individual chemical formulas, and also by name. In all such instances it is intended that the present invention include each individual stereoisomer of the compounds described herein, as well as racemic forms of the same.

The compounds of the present invention and their pharmaceutically acceptable salts are useful in for the treatment of viral infections in animal and human subjects, in particular HCV and SARS. The compounds of the invention can be used alone, or in a pharmaceutical composition containing one or more compounds of the invention, in combination with one or more pharmaceutically acceptable carriers. Thus, in further aspects, the present invention includes pharmaceutical compositions and methods of treating viral infections utilizing as an active ingredient the novel compounds described herein.

In some embodiments, the compounds of the invention can be prepared as salts, for example and not limitation, amine salts, which can contain any of a variety of pharmaceutically acceptable counterions. Suitable counterions for amine salts include acetate, adipate, aminosalicylate, anhydromethylenecitrate, ascorbate, aspartate, benzoate, benzenesulfonate, bromide, citrate, camphorate, camphorsulfonate, chloride, estolate, ethanesulfonate, fumarate, glucoheptanoate, gluconate, glutamate, lactobionate, malate, maleate, mandelate, methanesulfonate, pantothenate, pectinate, phosphate/diphosphate, polygalacturonate, propionate, salicylate, stearate, succinate, sulfate, tartrate and tosylate. Other suitable anionic species will be apparent to the skilled practitioner.

The compounds of the invention can be formulated in pharmaceutical compositions that can include one or more compounds of the invention and one or more pharmaceutically acceptable carriers. The compounds of the invention can be administered in powder or crystalline form, in liquid solution, or in suspension. They may be administered by a variety of means known to be efficacious for the administration of antiviral agents, including without limitation topically, orally and parenterally by injection (e.g., intravenously or intramuscularly).

When administered by injection, a preferred route of delivery for compounds of the invention is a unit dosage form in ampules, or in multidose containers. The injectable compositions may take such forms as suspensions, solutions, or emulsions in oily or aqueous vehicles, and may contain various formulating agents. Alternatively, the active ingredient may be in powder (lyophillized or non-lyophillized) form for reconstitution at the time of delivery with a suitable vehicle, such as sterile water. In injectable compositions, the carrier is typically comprised of sterile water, saline or another injectable liquid, e.g., peanut oil for intramuscular injections. Also, various buffering agents, preservatives and the like can be included.

Topical applications may be formulated in carriers such as hydrophobic or hydrophilic bases to form ointments, creams, lotions, in aqueous, oleaginous or alcoholic liquids to form paints or in dry diluents to form powders.

Oral compositions may take such forms as tablets, capsules, oral suspensions and oral solutions. The oral compositions may utilize carriers such as conventional formulating agents, and may include sustained release properties as well as rapid delivery forms.

The dosage to be administered depends to a large extent upon the condition and size of the subject being treated, the route and frequency of administration, the sensitivity of the pathogen to the particular compound selected, the virulence of the infection and other factors. Such matters, however, are left to the routine discretion of the physician according to principles of treatment well known in the antiviral arts. Another factor influencing the precise dosage regimen, apart from the nature of the infection and peculiar identity of the individual being treated, is the molecular weight of the compound.

The invention described herein also includes a method of treating a viral infection comprising administering to said mammal a compound of the invention in an amount effective to treat said infection. One preferred method of administration of the antiviral compounds of the invention include oral and parenteral, e.g., i.v. infusion, i.v. bolus and i.m. injection.

Compounds provided herein can be formulated into pharmaceutical compositions by admixture with pharmaceutically acceptable nontoxic excipients and carriers. As noted above, such compositions may be prepared for use in parenteral administration, particularly in the form of liquid solutions or suspensions; or oral administration, particularly in the form of tablets or capsules; or intranasally, particularly in the form of powders, nasal drops, or aerosols; or dermally, via, for example, transdermal patches; or prepared in other suitable fashions for these and other forms of administration as will be apparent to those skilled in the art.

The composition may conveniently be administered in unit dosage form and may be prepared by any of the methods well known in the pharmaceutical art, for example, as described in Remington's Pharmaceutical Sciences (Mack Pub. Co., Easton, Pa., 1980). Formulations for parenteral administration may contain as common excipients sterile water or saline, polyalkylene glycols such as polyethylene glycol, oils and vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of the active compounds. Other potentially useful parenteral delivery systems for these active compounds include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene-9-lauryl ether, glycocholate and deoxycholate, or oily solutions for administration in the form of nasal drops, or as a gel to be applied intranasally. Formulations for parenteral administration may also include glycocholate for buccal administration, a salicylate for rectal administration, or citric acid for vaginal administration. Formulations for transdermal patches are preferably lipophilic emulsions.

The materials of this invention can be employed as the sole active agent in a pharmaceutical or can be used in combination with other active ingredients, e.g., other agents useful in the treatment of viral infections.

The concentrations of the compounds described herein in a therapeutic composition will vary depending upon a number of factors, including the dosage of the drug to be administered, the chemical characteristics (e.g., hydrophobicity) of the compounds employed, and the route of administration. The compositions for human delivery per unit dosage, whether liquid or solid, may contain from about 0.01% to as high as about 99/% active material, the preferred range being from about 0.1%-60%. For example, the compounds of this invention may be provided in effective inhibitory amounts in an aqueous physiological buffer solution containing about 0.1 to 10% w/v compound for parenteral administration.

Typical dose ranges are from about 1 mg/kg to about 1 g/kg of body weight per day; a preferred dose range is from about 0.01 mg/kg to 100 mg/kg of body weight per day. Such formulations typically provide inhibitory amounts of the compound of the invention. The preferred dosage of drug to be administered is likely, however, to depend on such variables as the type and extent of progression of the disease or disorder, the overall health status of the particular patient, the relative biological efficacy of the compound selected, and formulation of the compound excipient, and its route of administration.

While the present invention has been described with specificity in accordance with certain of its preferred embodiments, the following examples serve only to illustrate the invention and are not intended to limit the same.

Nomenclature for these compounds was provided using ACD Name version 5.04 software (May 28, 2001) available from Advanced Chemistry Development, Inc and ChemInnovation NamExpert+Nomenclator™ brand software available from ChemInnovation Software, Inc. Some of the starting materials were named using standard IUPAC nomenclature.

### EXAMPLES

### Example 1

### Preparation of 3-methyl-4-propoxybenzaldeyhde

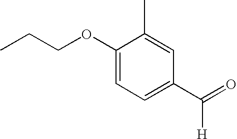

4-Hydroxy-3-methylbenzaldehyde [(1); 16.65 g; 1 eq], 1-bromopropane [12.2 ml; 1.1 eq], sodium iodide [4.58 g; 0.25 eq], potassium carbonate [33.8 g; 2 eq], and DMF (300 mL) were added to a dry round bottom flask. The flask was capped with a condenser and rubber septum then flushed with argon. The reaction was heated to 75° C. for 12 hours under an argon atmosphere. The reaction was then poured into a seperatory funnel containing water (900 mL) and ethyl acetate (900 mL). The organic layer was washed twice more with water and twice with brine, and dried over sodium sulfate. The organic filtrate was concentrated in vacuo to yield 3-methyl-4-propoxybenzaldeyhde as a crude oil. The product was used in the next step without further purification.

### Example 2

### Preparation of (3S)-3-{[(3-methyl-4-propoxyphenyl)methyl]amino}azaperhydroepin-2-one

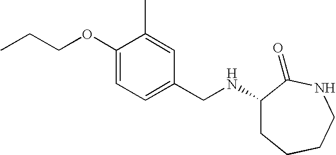

3-methyl-4-propoxybenzaldeyhde (9.72 g; 1 eq), (S)-alpha-amino-omega-caprolactam [6.99 g; 1 eq], sodium triacetoxyborohydride (34.68 g; 3 eq), and methylene chloride (220 mL) were added to a round bottom flask. The reaction was then stirred at room temperature for 5 hours. Saturated, aqueous sodium bicarbonate (200 mL) was carefully added to quench the reaction. The resulting mixture was diluted with ethyl acetate (200 mL) and shaken in a separatory funnel. After the organic layers were isolated, the aqueous layer was back extracted with two more portions of ethyl acetate (400 mL total). The organic layers were combined and dried over sodium sulfate. The sodium sulfate was filtered off and the organic filtrate was concentrated in vacuo to yield crude product. The resulting crude oil was dissolved in methylene chloride and purified via flash chromatography using a methylene chloride/methanol gradient. The pure fractions were combined and concentrated in vacuo to yield (3S)-3-{[(3-methyl-4-propoxyphenyl)methyl]amino}azaperhydroepin-2-one as an oil.

### Example 3

### Preparation of N-((3S)-2-oxoazaperhydroepin-3-yl)-2-4-carbonylphenoxy)-N-[(3-methyl-4-propoxyphenyl)methyl]acetamide

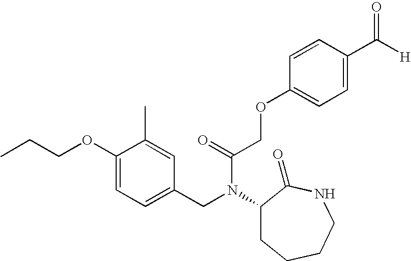

(3S)-3-{[(3-methyl-4-propoxyphenyl)-methyl]amino}azaperhydroepin-2-one (4.00 g), 4-formylphenoxyacetic acid (4.96 g; 2 eq), EDCI (5.28 g; 2 eq), and THF (30 mL) were added to a round bottom flask. The reaction was then stirred at room temperature for 5 hours. Afterwards, the mixture was concentrated in vacuo and diluted with ethyl acetate (50 ml) and water (50 ml). The resulting slurry was placed in a separatory funnel and shaken vigorously. The organic layers were then isolated and dried over sodium sulfate. The sodium sulfate was filtered off and the organic filtrate was concentrated in vacuo to yield crude product. The resulting impure solid was dissolved in methylene chloride and purified via flash chromatography using a methylene chloride/methanol gradient. The pure fractions were combined and concentrated in vacuo to yield N-((3S)-2-oxoazaperhydroepin-3-yl)-2-(4-carbonylphenoxy)-N-[(3-methyl-4-propoxyphenyl)methyl]acetamide as a pure solid.

### Example 4

### Preparation of N-((3S)-2-oxoazaperhydroepin-3-yl)-2-{4-[(cyclohexylamino)methyl]-phenoxy}-N-[(3-methyl-4-propoxyphenyl)methyl]acetamide

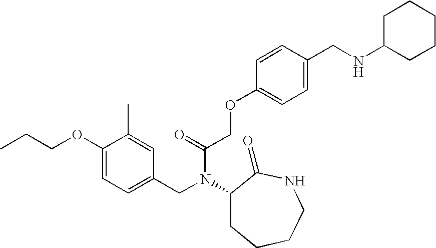

N-((3S)-2-oxoazaperhydroepin-3-yl)-2-(4-carbonylphenoxy)-N-[(3-methyl-4-propoxyphenyl)methyl]acetamide (0.30 g; 1 eq), cyclohexylamine (0.30 mL; 4 eq), and methylene chloride (3 mL) were added to a round bottom flask. The reaction was stirred at room temperature for 10 hours. Sodium triacetoxyborohydride (0.42 g; 3 eq) was then added to the mixture. The reaction was then stirred at room temperature for an additional 3 hours. Saturated, aqueous sodium bicarbonate (3 mL) was carefully added to quench the reaction. The resulting mixture was diluted with ethyl acetate (10 mL) and shaken in a separatory funnel. After the organic layers were isolated, the aqueous layer was back extracted with two more portions of ethyl acetate (20 mL total). The organic layers were combined and dried over sodium sulfate. The sodium sulfate was filtered off and the organic filtrate was concentrated in vacuo to yield crude solid. The solid mixture was then dissolved in DMSO and purified via prep HPLC. The pure fractions were then combined and lyophilized to yield N-((3S)-2-oxoazaperhydroepin-3-yl)-2-{4-[(cyclohexylamino)methyl]phenoxy}-N-[(3-methyl-4-propoxyphenyl)methyl]acetamide as a pure, white TFA salt.

### Example 5

### Preparation of N-((3S)-2-oxoazaperhydroepin-3-yl)-N-{1[3-(N-cyclohexylearbamoyl)-4-propoxyphenyl]methyl}-2-phenoxyacetamide (8)

This compound was synthesized according to Scheme 2 below:

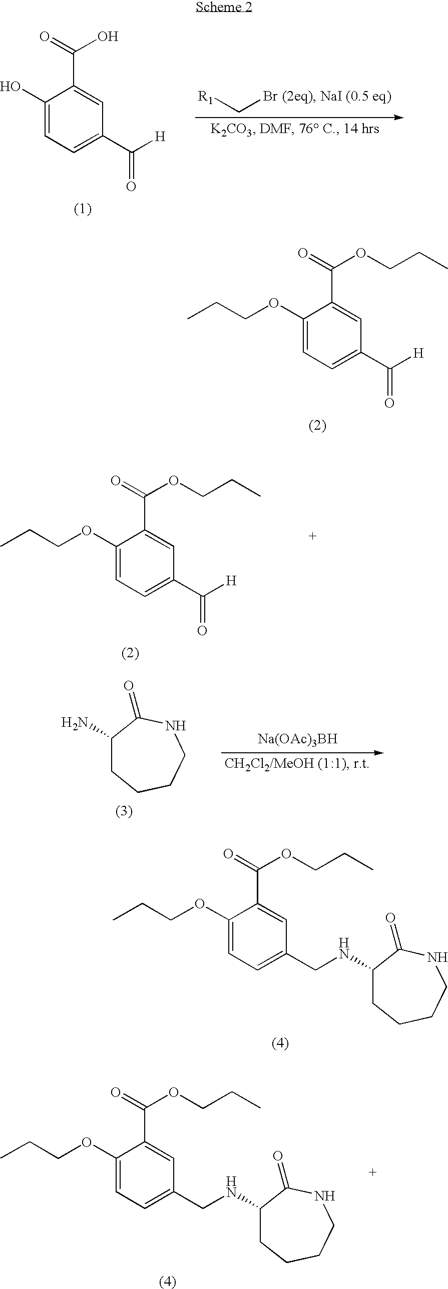

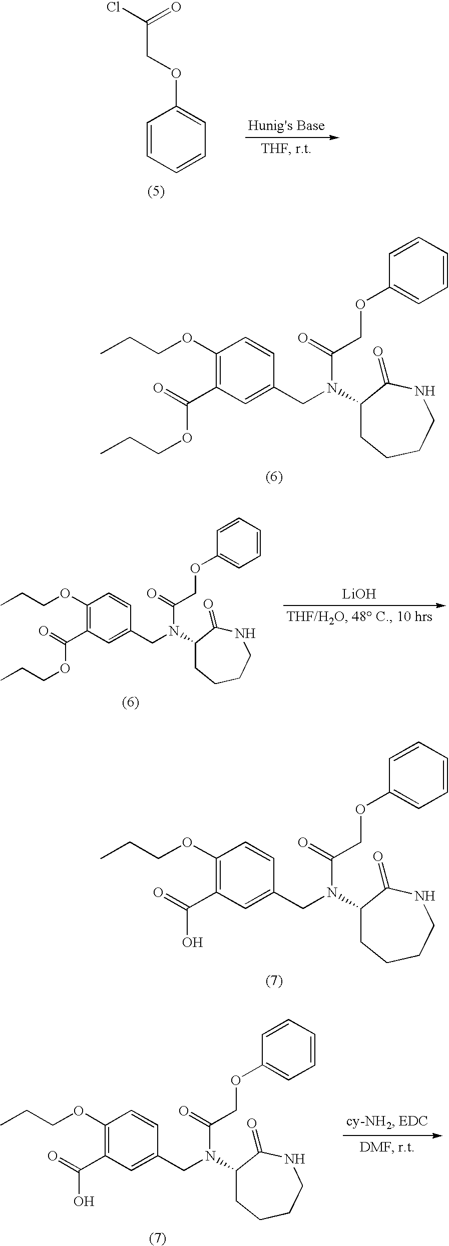

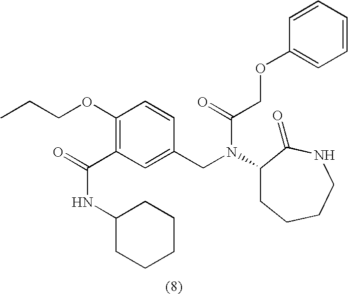

### a) 3-Methyl-4-propoxybenzaldeyhde (2)

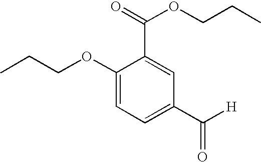

5-Formylsalicylic acid (8.76 g; 1 eq), 1-bromopropane (10.1 mL; 2.1 eq), sodium iodide (3.95 g; 0.5 eq), potassium carbonate (21.87 g; 3 eq), and DMF (211 mL) were added to a dry round bottom flask. The flask was capped with a condenser and rubber septum then flushed with argon. The reaction was heated to 75° C. for 12 hours under an argon atmosphere. The reaction was then poured into a seperatory funnel containing water (900 mL) and ethyl acetate (900 mL). The organic layer was washed twice more with water and twice with brine. The organic layer was isolated and dried over sodium sulfate. The sodium sulfate was filtered off and the organic filtrate was concentrated in vacuo to yield propyl 5-carbonyl-2-propoxybenzoate (1). The crude product was used in the next step without further purification.

### b) Propyl 5-{[((3S)-2-oxoazaperhydroepin-3-yl)amino]methyl}-2-propoxybenzoate (4)

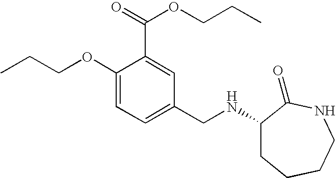

3-Methyl-4-propoxybenzaldeyhde (6.00 g; 1 eq), (S)-alpha-amino-omega-caprolactam (3.69 g; 1.2 eq), sodium triacetoxyborohydride (15.24 g, 3 eq), and methylene chloride (240 ml) were added to a round bottom flask. The reaction was then stirred at room temperature for 5 hours. Saturated, aqueous sodium bicarbonate (200 mL) was carefully added to quench the reaction. The resulting mixture was diluted with ethyl acetate (200 mL) and shaken in a separatory funnel. After the organic layers were isolated, the aqueous layer was back extracted with two more portions of ethyl acetate (400 mL total). The organic layers were combined and dried over sodium sulfate. The sodium sulfate was filtered off and the organic filtrate was concentrated in vacuo to yield crude product. The resulting crude oil was dissolved in methylene chloride and purified via flash chromatography using a methylene chloride/methanol gradient. The pure fractions were combined and concentrated in vacuo to yield propyl 5-{[((3S)-2-oxoazaperhydroepin-3-yl)amino]methyl}-2-propoxybenzoate as an oil.

### c) Propyl 5{[N-((3S)-2-oxoazaperhydroepin-3-yl)-2-phenoxyacetylamino]-methyl}-2-propoxybenzoate (6)

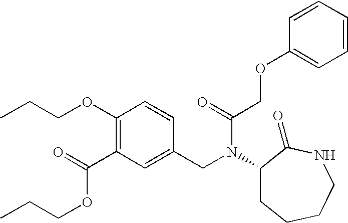

Propyl 5-{[((3S)-2-oxoazaperhydroepin-3-yl)amino]methyl}-2-propoxybenzoate (4.52 g; 1 eq), phenoxyacetyl chloride (2.1 mL; 1.2 eq), Hunig's base (2.6 mL; 1.2 eq], and THF (125 ml) were added to a round bottom flask. The reaction was then stirred at room temperature for 30 minutes. The reaction was then quenched by water addition (3 mL) and concentrated in vacuo to remove THF. The crude mixture was dissolved in ethyl acetate (200 mL) and washed with water (100 mL). The organic layer was then isolated and dried over sodium sulfate. The sodium sulfate was filtered off and the organic filtrate was concentrated in vacuo to yield crude product. The resulting crude oil was dissolved in methylene chloride and purified via flash chromatography using a methylene chloride 1 methanol gradient. The pure fractions were combined and concentrated in vacuo to yield propyl 5-{[N-((3S)-2-oxoazaperhydroepin-3-yl)-2-phenoxyacetylamino]-methyl}-2-propoxybenzoate (6) as pure solid.

### d) 5-{[N-((3S)-2-ooazaperhydroepin-3-yl)-2-phenoxyacetylamino]methyl}-2-propoxybenzoic acid (7)

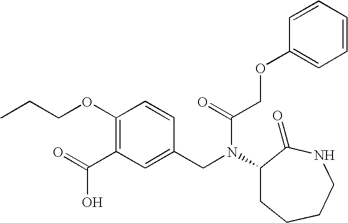

Propyl 5-{[N-((3S)-2-xoazaperhydroepin-3-yl)-2-phenoxyacetylamino]-methyl}-2-propoxybenzoate (1.15 g; 1 eq), lithium hydroxide (0.278 g; 5 eq), THF (11.5 ml), and water (1.15 mL) were added to a round bottom flask. The flask was fitted with a condenser and heated to 48° C. Once reaction was complete (roughly 18 hours —progress monitored by TLC every hour), the mixture was concentrated in vacuo to remove THF. The reaction was acidified to pH 6 by addition of 0.5 M aqueous citric acid. The precipitant was filtered off, washed with water, and dried by air suction to yield 5-{[N-((3S)-2-oxoazaperhydroepin-3-yl)-2-phenoxyacetylamino]methyl}-2-propoxybenzoic acid (7) as a pure white solid.

### e) N-((3S)-2-oxoazaperhydroepin-3-yl)-N-{[3-(N-cyclohexylcarbamoyl)-4-propoxyphenyl]methyl}-2-phenoxyacetamide (8)

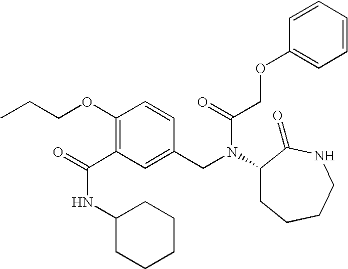

5-{[N-((3S)-2-oxoazaperhydroepin-3-yl)-2-phenoxyacetylamino]methyl}-2-propoxybenzoic acid (0.020 g; 1 eq), cyclohexyl amine (10.0 uL; 2 eq), EDCI (0.017 g; 2 eq), and DMF (0.5 ml) were added to a vial. The reaction was stirred at room temperature for 10 hours. The mixture was then filtered through a plug to remove insoluble particulates. The crude solution was directly injected into a preparatory HPLC. The pure fractions were combined and concentrated in vacuo to yield N-((3S)-2-oxoazaperhydroepin-3-yl)-N-{[3-(N-cyclohexylcarbamoyl)-4-propoxyphenyl]methyl}-2-phenoxyacetamide (8) as a pure solid.

### Example 6

### Preparation of N-((3S)-2-oxoazaperhydroepin-3-yl)-2-bromo-N-[(3-methyl-propoxyphenyl)methyl]acetamide

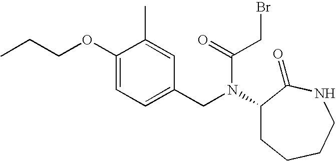

(3S)-3-{[(3-methyl-4-propoxyphenyl)methyl]amino}azaperhydroepin-2-one (3.00 g; 1 eq), Hunig's base (2.4 mL, 1.1 eq), and THF (100 ml) were added to a dry round bottom flask. The mixture was then cooled under an argon atmosphere to 0° C. using an ice/water bath. Bromoacetyl chloride (1.3 mL; 1.5 eq) was then added drop-wise to the above cold mixture over 5 minutes. The resulting solution was stirred for an additional 30 minutes at 0° C. The reaction was then quenched by water addition (3 ml) and concentrated in vacuo to remove THF. The crude mixture was dissolved in ethyl acetate (200 mL) and washed with water (100 mL). The organic layer was then isolated and dried over sodium sulfate. The sodium sulfate was filtered off and the organic filtrate was concentrated in vacuo to yield crude product. The resulting crude oil was dissolved in methylene chloride and purified via flash chromatography using a methylene chloride/methanol gradient. The pure fractions were combined and concentrated in vacuo to yield N-((3S)-2-oxoazaperhydroepin-3-yl)-2-bromo-N-[(3-methyl-4-propoxyphenyl)methyl]acetamide as pure solid.

### Example 7

### Preparation of N-((3S)-2-oxoazaperhydroepin-3-yl)-N-[(3-methyl-propoxyphenyl)methyl]-2-(phenylamino)acetamide

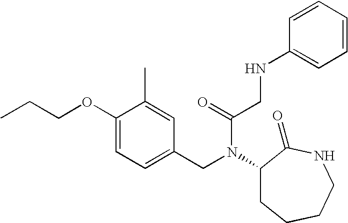

N-((3S)-2-oxoazaperhydroepin-3-yl)-2-bromo-N-[(3-methyl-4-propoxyphenyl)methyl]acetamide (0.250 g; 1 eq), aniline (0.11 ml; 2 eq), sodium iodide (0.023 g; 0.25 eq), potassium carbonate (0.168 g; 2 eq), and DMF (2.4 ml) were added to a round bottom flask. The reaction was then heated to 75° C. for 8 hours under an argon atmosphere. After cooling to room temperature, the mixture was filtered through a cotton plug to remove excess insoluble salts, namely sodium iodide and potassium carbonate. The crude solution was purified by prep-HPLC. The pure fractions were combined and concentrated in vacuo to yield N-((3S)-2-oxoazaperhydroepin-3-yl)-N-[(3-methyl-4-propoxyphenyl)methyl]-2-(phenylamino)acetamide as a TFA salt.

As will be appreciated, the general procedures of Example 7 can be employed to prepare a wide variety of compound of the invention. For example, the bromide of Example 7 can be displaced by any of a wide a variety of nucleophiles such as anilines, thiophenols, alkoxides, etc., using the same general conditions.

### Examples 8-82

### Representative Substituted Oxoazepanylacetamide Compounds

Representative substituted oxoazepanylacetamide compounds of the invention are shown in Table 2. In Table 2, MH+ refers to the molecular ion observed by mass spectrometry.

TABLE 2
Representative Substituted Oxoazepanylacetamides

| Ex- | | | |
|---|---|---|---|
| am- | | | |
| ple | Structure | Name | MH+ |
| 8 | 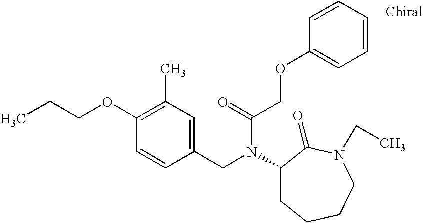 | N-[(3S)-1-ethyl-2-oxoazepan-3-yl]-N-(3-methyl-4-propoxybenzyl)-2-phenoxyacetamide | 453.3 |
| 9 | 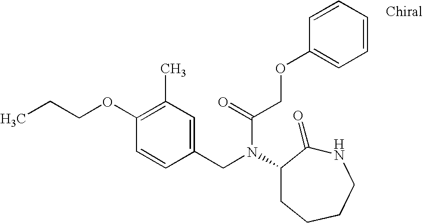 | N-(3-methyl-4-propoxybenzyl)-N-[(3S)-2-oxoazepan-3-yl]-2-phenoxyacetamide | 425.2 |
| 10 | 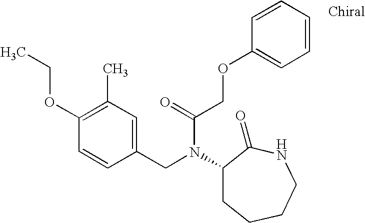 | N-(4-ethoxy-3-methylbenzyl)-N-[(3S)-2-oxoazepan-3-yl]-2-phenoxyacetamide | 411.2 |
| 11 | 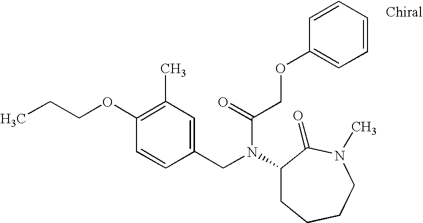 | N-[(3S)-1-methyl-2-oxoazepan-3-yl]-N-(3-methyl-4-propoxybenzyl)-2-phenoxyacetamide | 439.2 |
| 12 | 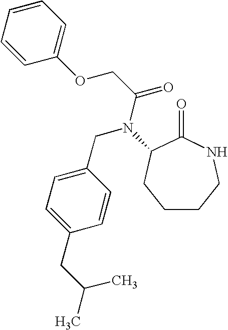 | N-(4-isobutylbenzyl)-N-[(3S)-2-oxoazepan-3-yl]-2-phenoxyacetamide | 409.2 |
| 13 | 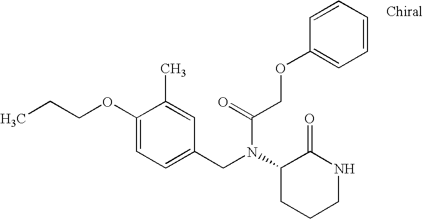 | N-(3-methyl-4-propoxybenzyl)-N-[(3S)-2-oxopiperidin-3-yl]-2-phenoxyacetamide | 411.2 |
| 14 | 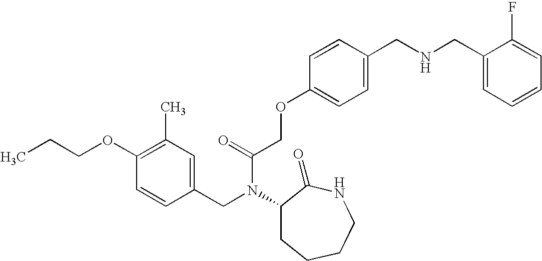 | 2-(4-{[(2-fluorobenzyl)amino]methyl}phenoxy)-N-(3-methyl-4-propoxybenzyl)-N-[(3S)-2-oxoazepan-3-yl]acetamide | 562.3 |
| 15 | 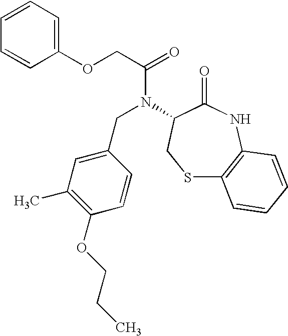 | N-(3-methyl-4-propoxybenzyl)-N-[(3R)-4-oxo-2,3,4,5-tetrahydro-1,5-benzothiazepin-3-yl]-2-phenoxyacetamide | 491.2 |
| 16 | 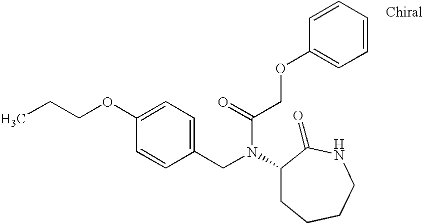 | N-[(3S)-2-oxoazepan-3-yl]-2-phenoxy-N-(4-propoxybenzyl)acetamide | 411.2 |
| 17 | 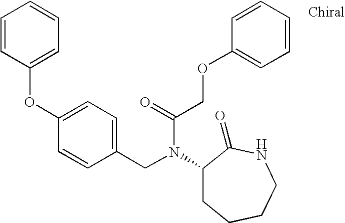 | N-[(3S)-2-oxoazepan-3-yl]-2-phenoxy-N-(4-phenoxybenzyl)acetamide | 445.2 |
| 18 | 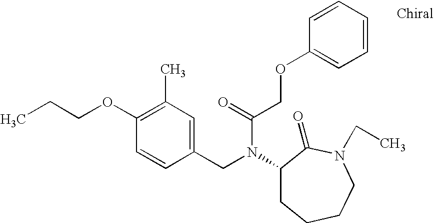 | 2-[4-(anilinomethyl)phenoxy]-N-(3-methyl-4-propoxybenzyl)-N-[(3S)-2-oxoazepan-3-yl]acetamide | 530.3 |
| 19 | 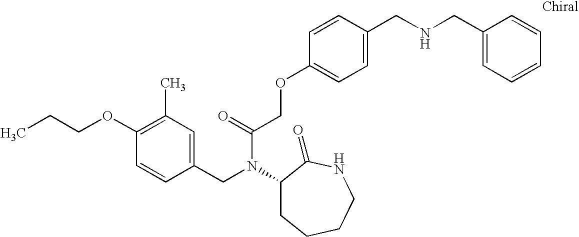 | 2-{4-[(benzylamino)methyl]phenoxy}-N-(3-methyl-4-propoxybenzyl)-N-[(3S)-2-oxoazepan-3-yl]acetamide | 544.4 |
| 20 | 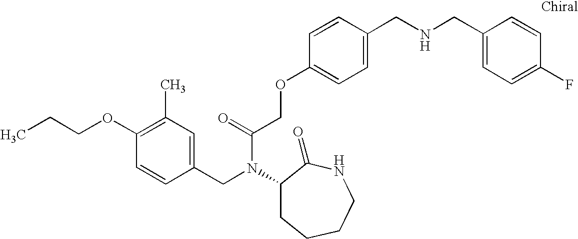 | 2-(4-{[(4-fluorobenyzl)amino]methyl}phenoxy)-N-(3-methyl-4-propoxybenzyl)-N-[(3S)-2-oxoazepan-3-yl]acetamide | 562.3 |
| 21 | 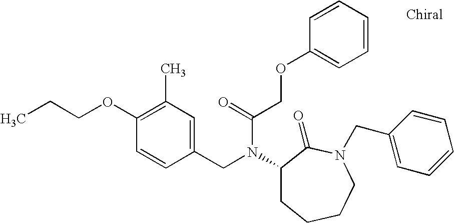 | N-[(3S)-1-benzyl-2-oxoazepan-3-yl]-N-(3-methyl-4-propoxybenzyl)-2-phenoxyacetamide | 515.3 |
| 22 | 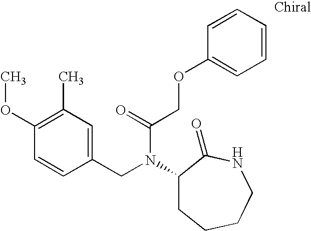 | N-(4-methoxy-3-methylbenzyl)-N-[(3S)-2-oxoazepan-3-yl]-2-phenoxyacetamide | 397.2 |
| 23 | 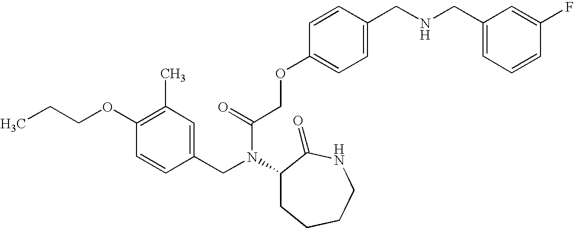 | 2-(4-{[(3-fluorobenzyl)amino]methyl}phenoxy)-N-(3-methyl-4-propoxybenzyl)-N-[(3S)-2-oxoazepan-3-yl]acetamide | 562.3 |
| 24 | 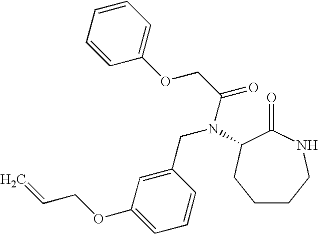 | N-[3-(allyloxy)benzyl]-N-[(3S)-2-oxoazepan-3-yl]-2-phenoxyacetamide | 409.2 |
| 25 | 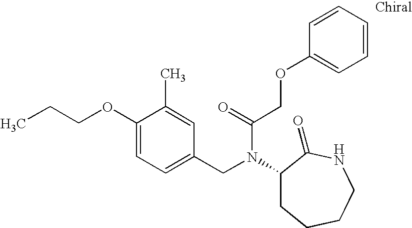 | N˜1˜-(3-methyl-4-propoxybenzyl)-N˜1˜-[(3S)-2-oxoazepan-3-yl]-N˜-2-phenylglycinamide | 424.2 |
| 26 | 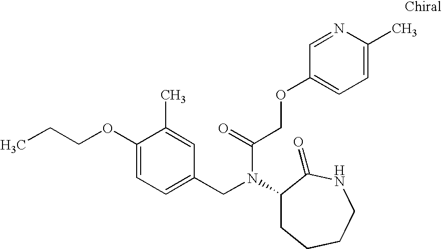 | N-(3-methyl-4-propoxybenzyl)-2-[(6-methylpyridin-3-yl)oxy]-N-[(3S)-2-oxoazepan-3-yl]acetamide | 440 |
| 27 | 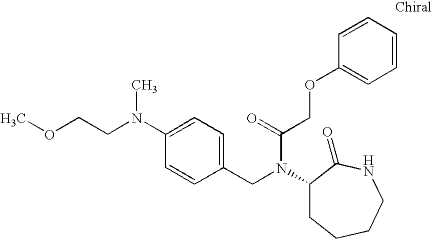 | N-{4-[(2-methoxyethyl)(methyl)amino]benzyl}-N-[(3S)-2-oxoazepan-3-yl]-2-phenoxyacetamide | 440.2 |
| 28 | 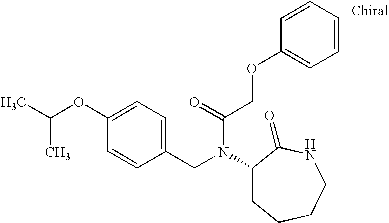 | N-(4-isopropoxybenzyl)-N-[(3S)-2-oxoazepan-3-yl]-2-phenoxyacetamide | 411.2 |
| 29 | 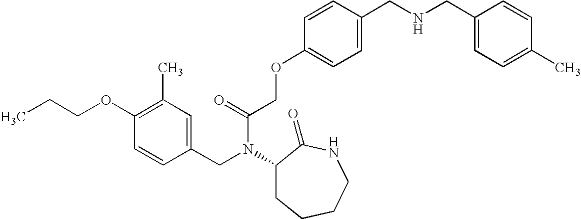 | 2-(4-{[(4-methylbenzyl)amino]methyl}phenoxy)-N-(3-methyl-4-propoxybenzyl)-N-[(3S)-2-oxoazepan-3-yl]acetamide | 558.3 |
| 30 | 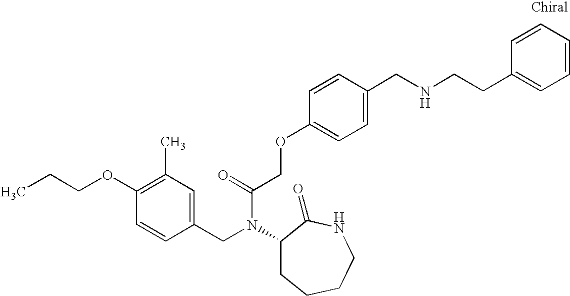 | N-(3-methyl-4-propoxybenzyl)-N-[(3S)-2-oxoazepan-3-yl]-2-(4-{[(2-phenylethyl)amino]methyl}phenoxy)acetamide | 558.3 |
| 31 | 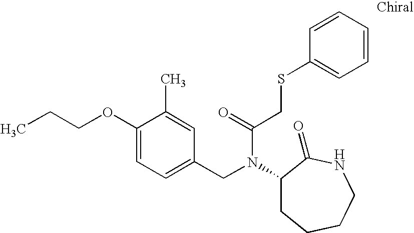 | N-(3-methyl-4-propoxybenzyl)-N-[(3S)-2-oxoazepan-3-yl]-2-(phenylthio)acetamide | 441.2 |
| 32 | 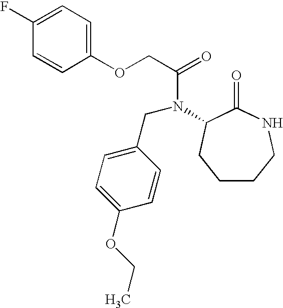 | N-(4-ethoxybenzyl)-2-(4-fluorophenoxy)-N-[(3S)-2-oxoazepan-3-yl]acetamide | 415.2 |
| 33 | 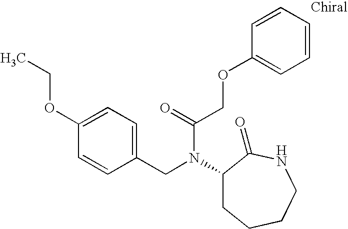 | N-(4-ethoxybenzyl)-N-[(3S)-2-oxoazepan-3-yl]-2-phenoxyacetamide | 397.2 |
| 34 | 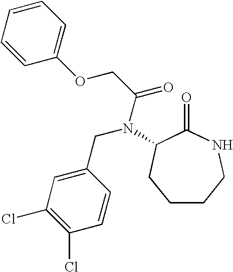 | N-(3,4-dichlorobenzyl)-N-[(3S)-2-oxoazepan-3-yl]-2-phenoxyacetamide | 421.1 |
| 35 | 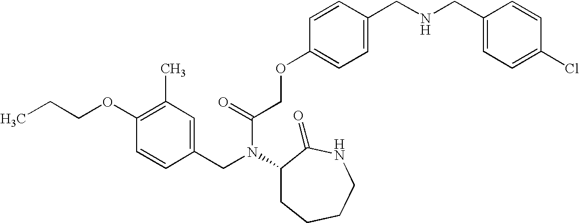 | 2-(4-{[(4-chlorobenzyl)amino]methyl}phenoxy)-N-(3-methyl-4-propoxybenzyl)-N-[(3S)-2-oxoazepan-3-yl]acetamide | 578.3 |
| 36 | 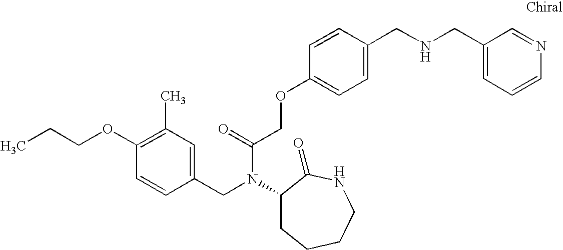 | N-(3-methyl-4-propoxybenzyl)-N-[(3S)-2-oxoazepan-3-yl]-2-(4-{[(pyridin-3-ylmethyl)amino]methyl}phenoxy)acetamide | 545.3 |
| 37 | 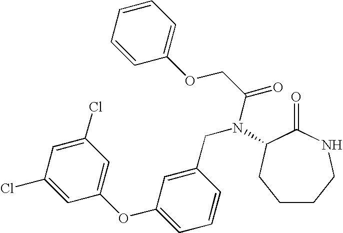 | N-[3-(3,5-dichlorophenoxy)benzyl]-N-[(3S)-2-oxoazepan-3-yl]-2-phenoxyacetamide | 513.1 |
| 38 | 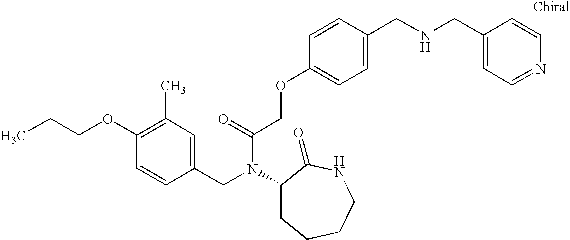 | N-(3-methyl-4-propoxybenzyl)-N-[(3S)-2-oxoazepan-3-yl]-2-(4-{[(pyridin-4-ylmethyl)amino]methyl}phenoxy)acetamide | 545.3 |
| 39 | 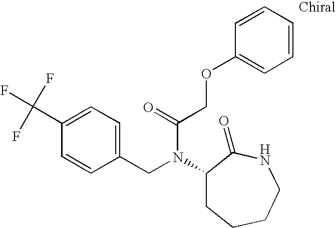 | N-[(3S)-2-oxoazepan-3-yl]-2-phenoxy-N-[4-(trifluoromethyl)benzyl]acetamide | 421.2 |
| 40 | 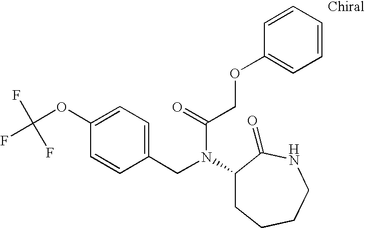 | N-[(3S)-2-oxoazepan-3-yl]-2-phenoxy-N-[4-(trifluoromethoxy)benzyl]acetamide | 437.2 |
| 41 | 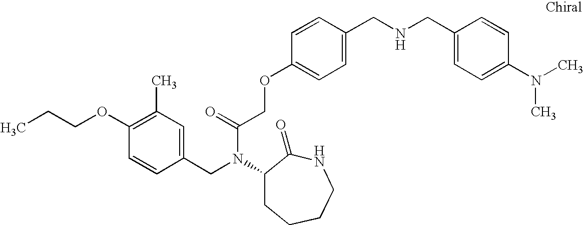 | 2-[4-({[4-(dimethylamino)benzyl]amino}methyl)phenoxy]-N-(3-methyl-4-propoxybenzyl)-N-[(3S)-2-oxoazepan-3-yl]acetamide | 587.4 |
| 42 | 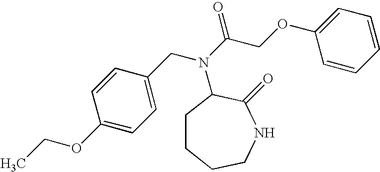 | N-(4-ethoxybenzyl)-N-(2-oxoazepan-3-yl)-2-phenoxyacetamide | 397.2 |
| 43 | 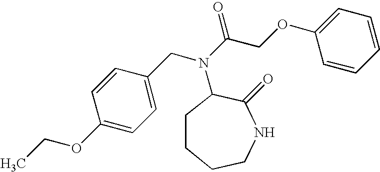 | N-(4-ethoxybenzyl)-N-(2-oxoazepan-3-yl)-2-phenoxyacetamide | 397.1 |
| 44 | 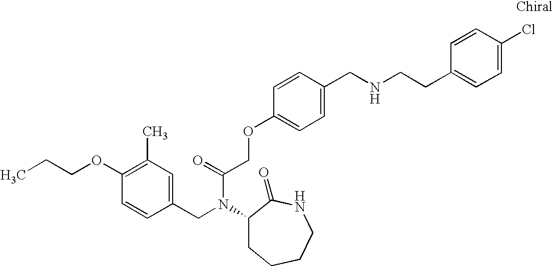 | 2-[4-({[2-(4-chlorophenyl)ethyl]amino}methyl)phenoxy]-N-(3-methyl-4-propoxybenzyl)-N-[(3S)-2-oxoazepan-3-yl]acetamide | 592.3 |
| 45 | 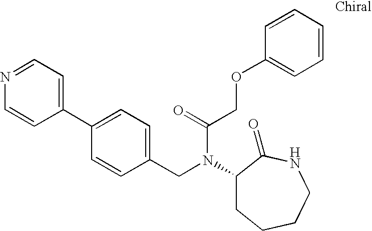 | N-[(3S)-2-oxoazepan-3-yl]-2-phenoxy-N-(4-pyridin-4-ylbenzyl)acetamide | 430.2 |
| 46 | 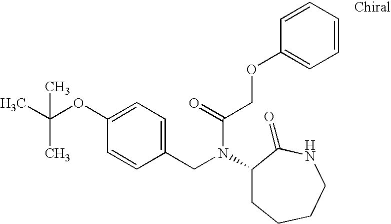 | N-(4-tert-butoxybenzyl)-N-[(3S)-2-oxoazepan-3-yl]-2-phenoxyacetamide | 425.2 |
| 47 | 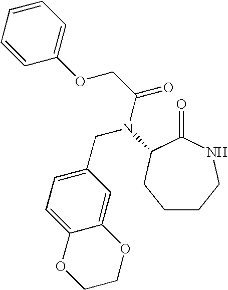 | N-(2,3-dihydro-1,4-benzodioxin-6-ylmethyl)-N-[(3S)-2-oxoazepan-3-yl]-2-phenoxyacetamide | 411.2 |
| 48 | 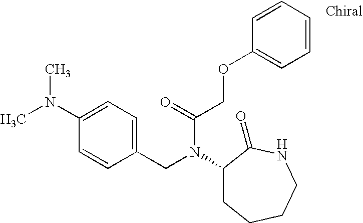 | N-[4-(dimethylamino)benzyl]-N-[(3S)-2-oxoazepan-3-yl]-2-phenoxyacetamide | 396.2 |
| 49 | 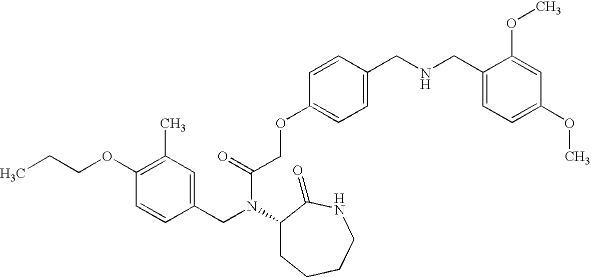 | 2-(4-{[(2,4-dimethoxybenzyl)amino]methyl}phenoxy)-N-(3-methyl-4-propoxybenzyl)-N-[(3S)-2-oxoazepan-3-yl]acetamide | 604.3 |
| 50 | 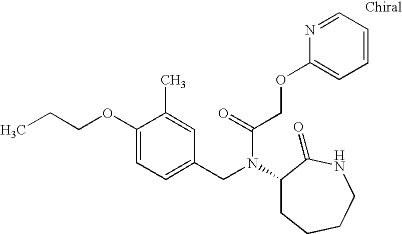 | N-(3-methyl-4-propoxybenzyl)-N-[(3S)-2-oxoazepan-3-yl]-2-(pyridin-2-yloxy)acetamide | 426.1 |
| 51 | 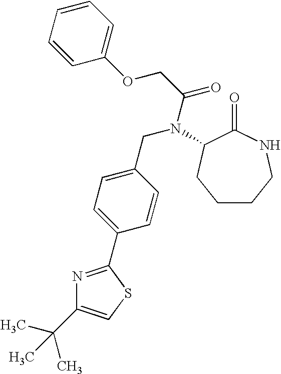 | N-[4-(4-tert-butyl-1,3-thiazol-2-yl)benzyl]-N-[(3S)-2-oxoazepan-3-yl]-2-phenoxyacetamide | 492.2 |
| 52 | 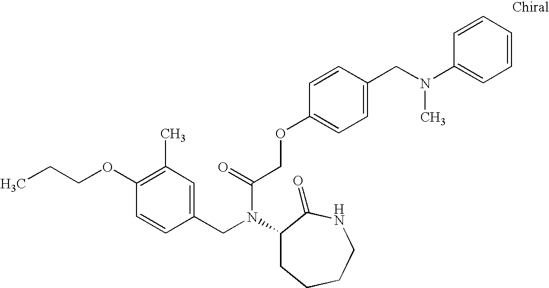 | 2-(4-{[methyl(phenyl)amino]methyl}phenoxy)-N-(3-methyl-4-propoxybenzyl)-N-[(3S)-2-oxoazepan-3-yl]acetamide | 544.3 |
| 53 | 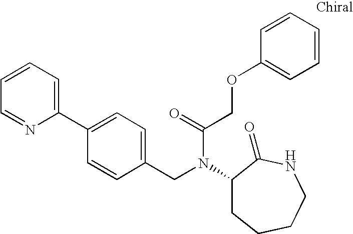 | N-[(3S)-2-oxoazepan-3-yl]-2-phenoxy-N-(4-pyridin-2-ylbenzyl)acetamide | 430.2 |
| 54 | 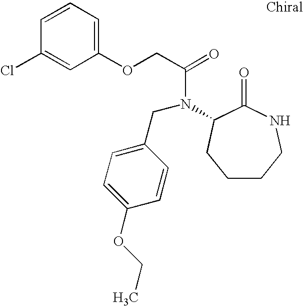 | 2-(3-chlorophenoxy)-N-(4-ethoxybenzyl)-N-[(3S)-2-oxoazepan-3-yl]-2-phenoxyacetamide | 431.2 |
| 55 | 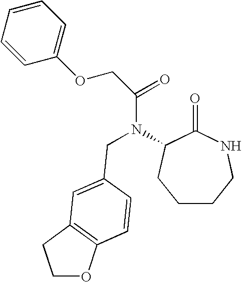 | N-(2,3-dihydro-1-benzofuran-5-ylmethyl)-N-[(3S)-2-oxoazepan-3-yl]-2-phenoxyacetamide | 395.2 |
| 56 | 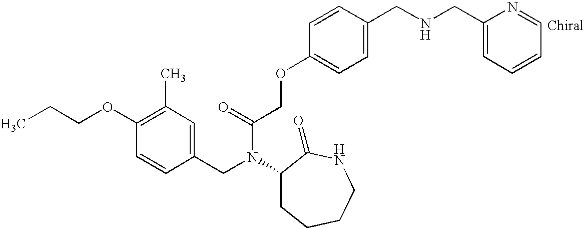 | N-(3-methyl-4-propoxybenzyl)-N-[(3S)-2-oxoazepan-3-yl]-2-(4-{[(pyridin-2-ylmethyl)amino]methyl}phenoxy)acetamide | 545.3 |
| 57 | 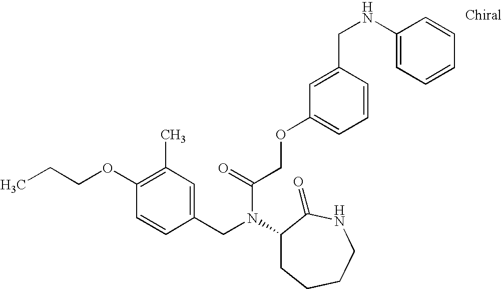 | 2-[3-(anilinomethyl)phenoxy]-N-(3-methyl-4-propoxybenzyl)-N-[(3S)-2-oxoazepan-3-yl]acetamide | 530.3 |
| 58 | 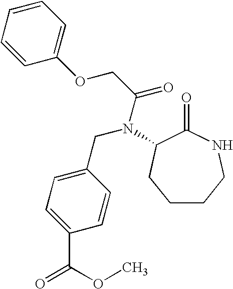 | methyl 4-{[[(3S)-2-oxoazepan-3-yl](phenoxyacetyl)amino]methyl}benzoate | 411.2 |
| 59 | 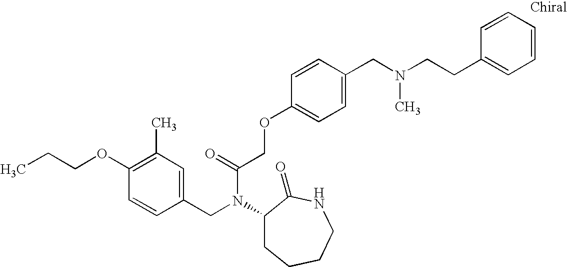 | 2-(4-{[methyl(2-phenylethyl)amino]methyl}phenoxy)-N-(3-methyl-4-propoxybenzyl)-N-[(3S)-2-oxoazepen-3-yl]acetamide | 572.3 |
| 60 | 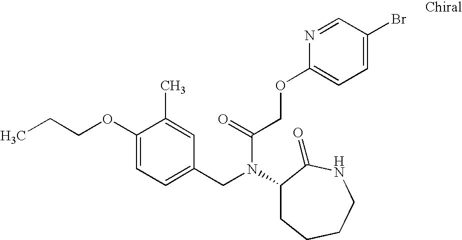 | 2-[(5-bromopyridin-2-yl)oxy]-N-(3-methyl-4-propoxybenzyl)-N-[(3S)-2-oxoazepan-3-yl]acetamide | 504.1 |
| 61 | 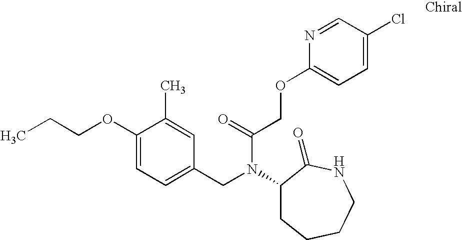 | 2-[(5-chloropyridin-2-yl)oxy]-N-(3-methyl-4-propoxybenzyl)-N-[(3S)-2-oxoazepan-3-yl]acetamide | 460.2 |
| 62 | 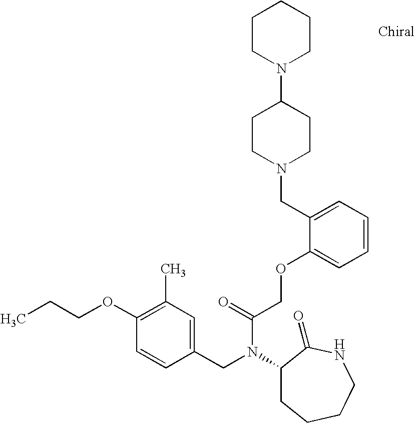 | 2-[2-(1,4′-bipiperidin-1′-ylmethyl)phenoxy]-N-(3-methyl-4-propoxybenzyl)-N-[(3S)-2-oxoazepan-3-yl]acetamide | 605.4 |
| 63 | 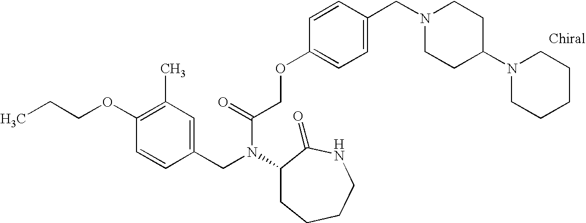 | 2-[4-(1,4′-bipiperidin-1′-ylmethyl)phenoxy]-N-(3-methyl-4-propoxybenzyl)-N-[(3S)-2-oxoazepan-3-yl]acetamide | 605.4 |
| 64 | 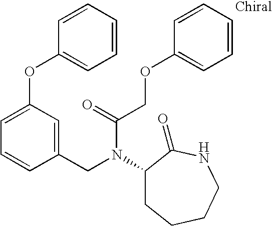 | N-[(3S)-2-oxoazepan-3-yl]-2-phenoxy-N-(3-phenoxybenzyl)acetamide | 445.2 |
| 65 | 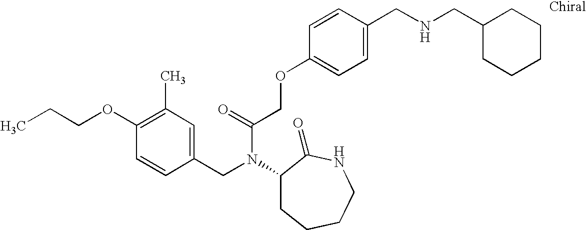 | 2-(4-{[(cyclohexylmethyl)amino]methyl}phenoxy)-N-(3-methyl-4-propoxy)-N-[(3S)-2-oxoazepan-3-yl]acetamide | 550.3 |
| 66 | 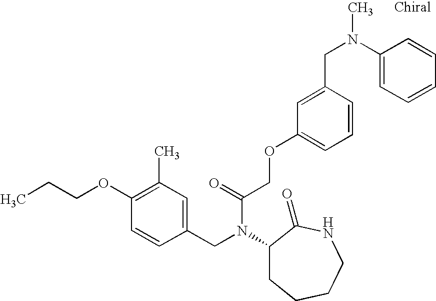 | 2-(3-{[methyl(phenyl)amino]methyl}phenoxy)-N-(3-methyl-4-propoxybenzyl)-N-[(3S)-2-oxoazepan-3-yl]acetamide | 544.3 |
| 67 | 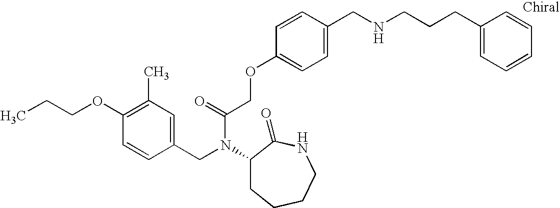 | N-(3-methyl-4-propoxybenzyl)-N-[(3S)-2-oxoazepan-3-yl]-2-(4-{[(3-phenylpropyl)amino]methyl}phenoxy)acetamide | 572.4 |
| 68 | 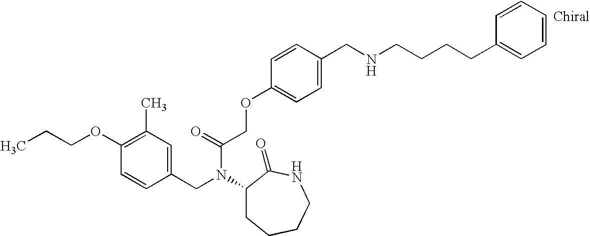 | N-(3-methyl-4-propoxybenzyl)-N-[(3S)-2-oxoazepan-3-yl]-2-(4-{[(4-phenylbutyl)amino]methyl}phenoxy)acetamide | 586.3 |
| 69 | 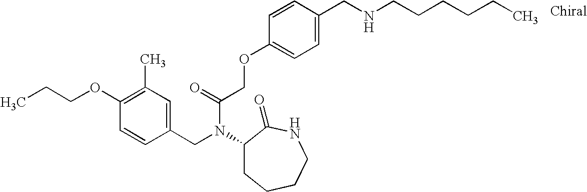 | 2-{4-[(hexylamino)methyl]phenoxy}-N-(3-methyl-4-propoxybenzyl)-N-[(3S)-2-oxoazepan-3-yl]acetamide | 538.3 |
| 70 | 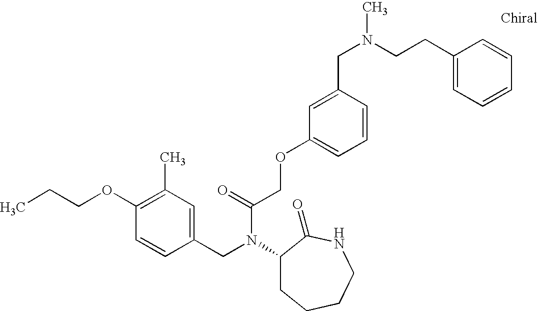 | 2-(3-{[methyl(2-phenylethyl)amino]methyl}phenoxy)-N-(3-methyl-4-propoxybenzyl)-N-[(3S)-2-oxoazepan-3-yl]acetamide | 572.3 |
| 71 | 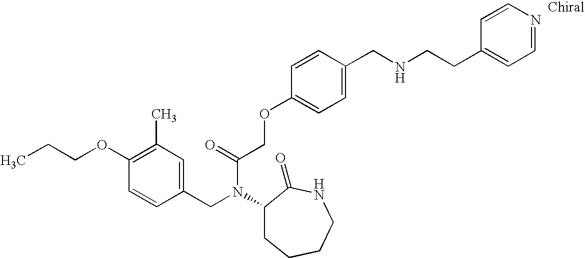 | N-(3-methyl-4-propoxybenzyl)-N-[(3S)-2-oxoazepan-3-yl]-2-(4-{[(2-pyridin-4-ylethyl)amino]methyl}phenoxy)acetamide | 559.3 |
| 72 | 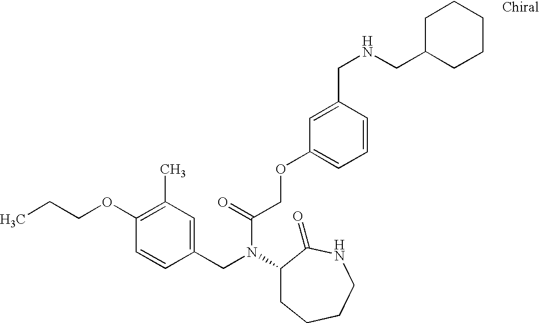 | 2-(3-{[(cyclohexylmethyl)amino]methyl}phenoxy)-N-(3-methyl-4-propoxybenzyl)-N-[(3S)-2-oxoazepan-3-yl]acetamide | 550.3 |
| 73 | 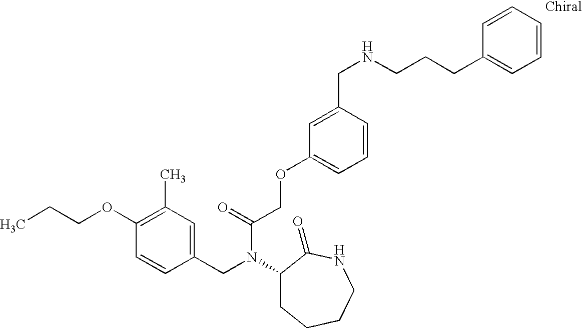 | N-(3-methyl-4-propoxybenzyl)-N-[(3S)-2-oxoazepan-3-yl]-2-(3-{[(3-phenylpropyl)amino]methyl}phenoxy)acetamide | 572.3 |
| 74 | 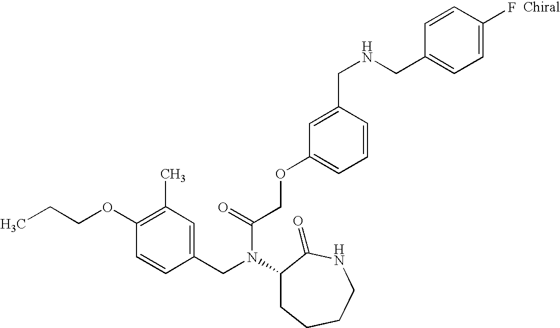 | 2-(3-{[(4-fluorobenzyl)amino]methyl}phenoxy)-N-(3-methyl-4-propoxybenzyl)-N-[(3S)-2-oxoazepan-3-yl]acetamide | 562.3 |
| 75 | 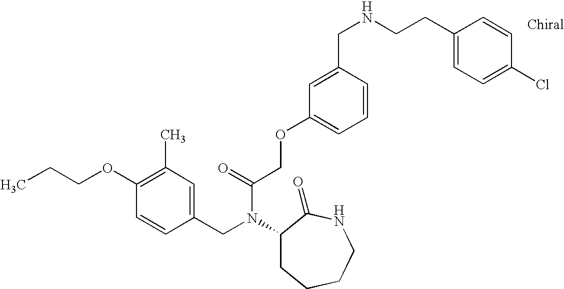 | 2-[3-({[2-(4-chlorophenyl)ethyl]amino}methyl}phenoxy]-N-(3-methyl-4-propoxybenzyl)-N-[(3S)-2-oxoazepan-3-yl]acetamide | 592.3 |
| 76 | 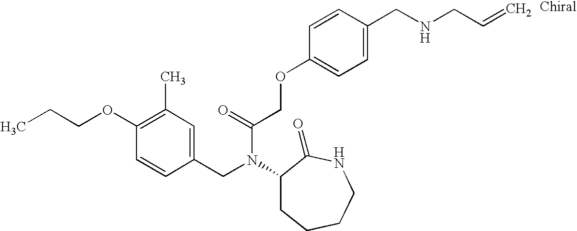 | 2-{4-[(allylamino)methyl]phenoxy}-N-(3-methyl-4-propoxybenzyl)-N-[(3S)-2-oxoazepan-3-yl]acetamide | 494.2 |
| 77 | 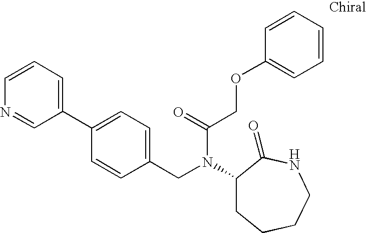 | N-[(3S)-2-oxoazepan-3-yl]-2-phenoxy-N-(4-pyridin-3-ylbenzyl)benzamide | 430.2 |
| 78 | 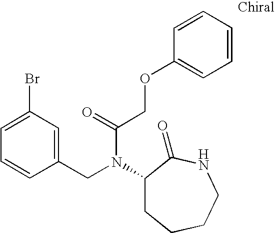 | N-(3-bromobenzyl)-N-[(3S)-2-oxoazepan-3-yl]-2-phenoxyacetamide | 431.1 |
| 79 | 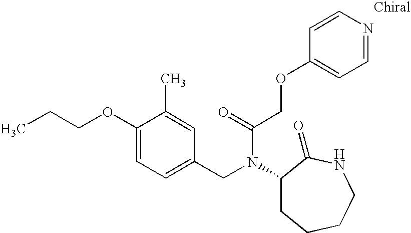 | N-(3-methyl-4-propoxybenzyl)-N-[(3S)-2-oxoazepan-3-yl]-2-(pyridin-4-yloxy)acetamide | 426.1 |
| 80 | 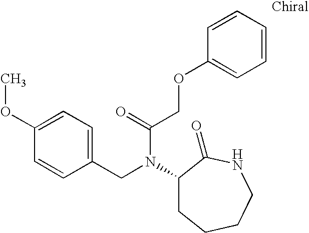 | N-(4-methoxybenzyl)-N-[(3S)-2-oxoazepan-3-yl]-2-phenoxyacetamide | 383.2 |
| 81 | 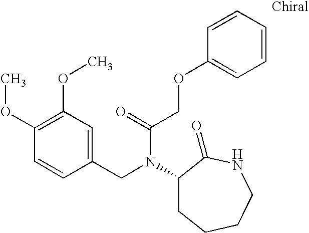 | N-(3,4-dimethoxybenzyl)-N-[(3S)-2-oxoazepan-3-yl]-2-phenoxyacetamide | 413.2 |
| 82 | 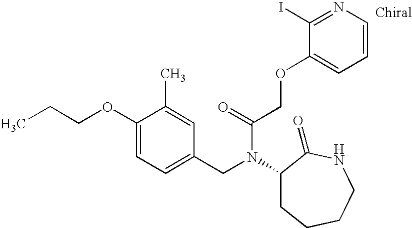 | 2-[(2-iodopyridin-3-yl)oxy]-N-(3-methyl-4-propoxybenzyl)-N-[(3S)-2-oxoazepan-3-yl]acetamide | 552.1 |

### Assay Procedures

### Example 83

### Quantification of HCV Replicon RNA in Cell Lines (HCV Cell Based Assay)

Cell lines, including Huh-11-7 or Huh 9-13, harboring HCV replicons (Lohmann, et al Science 285:110-113, 1999) are seeded at 5×10³ cells/well in 96 well plates and fed media containing DMEM (high glucose), 10% fetal calf serum, penicillin-streptomycin and non-essential amino acids. Cells are incubated in a 5% CO₂ incubator at 37° C. At the end of the incubation period, total RNA is extracted and purified from cells using Qiagen RNeasy 96 Kit (Catalog No. 74182). To amplify the HCV RNA so that sufficient material can be detected by an HCV specific probe (below), primers specific for HCV (below) mediate both the reverse transcription (RT) of the HCV RNA and the amplification of the cDNA by polymerase chain reaction (PCR) using the TaqMan One-Step RT-PCR Master Mix Kit (Applied Biosystems catalog no. 4309169). The nucleotide sequences of the RT-PCR primers, which are located in the NS5B region of the HCV genome, are the following:

HCV Forward primer “RBNS5bfor”:
5′ GCTGCGGCCTGTCGAGCT
HCV Reverse primer “RBNS5Brev”:
5′ GAAGGTCGTCTCCGCATAC

Detection of the RT-PCR product was accomplished using the Applied Biosystems (ABI) Prism 7700 Sequence Detection System (SDS) that detects the fluorescence that is emitted when the probe, which is labeled with a fluorescence reporter dye and a quencher dye, is processed during the PCR reaction. The increase in the amount of fluorescence is measured during each cycle of PCR and reflects the increasing amount of RT-PCR product. Specifically, quantification is based on the threshold cycle, where the amplification plot crosses a defined fluorescence threshold. Comparison of the threshold cycles of the sample with a known standard provides a highly sensitive measure of relative template concentration in different samples (ABI User Bulletin #2 Dec. 11, 1997). The data is analyzed using the ABI SDS program version 1.7. The relative template concentration can be converted to RNA copy numbers by employing a standard curve of HCV RNA standards with known copy number (ABI User Bulletin #2 Dec. 11, 1997).

The RT-PCR product was detected using the following labeled probe:

5′ FAM-CGAAGCTCCAGGACTGCACGATGCT-TAMRA
FAM = Fluorescence reporter dye.
TAMRA = Quencher dye.

The RT reaction is performed at 48° C. for 30 minutes followed by PCR. Thermal cycler parameters used for the PCR reaction on the ABI Prism 7700 Sequence Detection System were: one cycle at 95° C., 10 minutes followed by 35 cycles each of which included one incubation at 95° C. for 15 seconds and a second incubation for 60° C. for 1 minute.

To normalize the data to an internal control molecule within the cellular RNA, RT-PCR was performed on the cellular messenger RNA glyceraldehydes-3-phosphate dehydrogenase (GAPDH). The GAPDH copy number is very stable in the cell lines used. GAPDH RT-PCR is performed on the same exact RNA sample from which the HCV copy number is determined. The GAPDH primers and probes, as well as the standards with which to determine copy number, is contained in the ABI Pre-Developed TaqMan Assay Kit (catalog no. 4310884E). The ratio of HCV/GAPDH RNA is used to calculate the activity of compounds evaluated for inhibition of HCV RNA replication.

### Example 84

### Activity of Compounds as Inhibitors of HCV Replication (Cell based Assay) in Replicon Containing Huh-7 Cell Lines

The effect of a specific anti-viral compound on HCV replicon RNA levels in Huh-11-7 or 9-13 cells, cells was determined by comparing the amount of HCV RNA normalized to GAPDH (e.g. the ratio of HCV/GAPDH) in the cells exposed to compound versus cells exposed to the 0% inhibition and the 100% inhibition controls. Specifically, cells were seeded at 5×10³ cells/well in a 96 well plate and were incubated either with: 1) media containing 1% DMSO (0% inhibition control), 2) 100 international units, IU/ml Interferon-alpha 2b in media/1% DMSO or 3) media/1% DMSO containing a fixed concentration of compound. 96 well plates as described above were then incubated at 37° C. for 3 days (primary screening assay) or 4 days (IC50 determination). Percent inhibition was defined as:

% Inhibition=[100−((S−C2)/C1−C₂))]×100

where:
S=the ratio of HCV RNA copy number/GAPDH RNA copy number in the sample
C1=the ratio of HCV RNA copy number/GAPDH RNA copy number in the 0% inhibition control (media/1% DMSO)
C2=the ratio of HCV RNA copy number/GAPDH RNA copy number in the 100% inhibition control (100 IU/ml Interferon-alpha 2b)

The dose-response curve of the inhibitor was generated by adding compound in serial, three-fold dilutions over three logs to wells starting with the highest concentration of a specific compound at 10 uM and ending with the lowest concentration of 0.01 uM. Further dilution series (1 uM to 0.001 uM for example) was performed if the IC50 value was not in the linear range of the curve. IC50 was determined based on the IDBS Activity Base program using Microsoft Excel “XL Fit” in which A=100% inhibition value (100 IU/ml Interferon-alpha 2b), B=0% inhibition control value (media/1% DMSO) and C=midpoint of the curve as defined as C−(B−A/2)+A. A, B and C values are expressed as the ratio of HCV RNA/GAPDH RNA as determined for each sample in each well of a 96 well plate as described above. For each plate the average of 4 wells were used to define the 100% and 0% inhibition values.

Each of the compounds listed in Table 2, which can be synthesized using the procedures described in Scheme 1 and in Examples 1-7, can be assayed as described above in Example 83 and/or Example 84. Many of these compounds showed activity at less than 10 μM with respect to inhibition of HCV. Some of these compounds showed activity at less than 1 μM with respect to inhibition of HCV. More particularly, some compounds of Examples 1-82 showed inhibition of HCV at less than 0.1 μM. Thus, in some preferred embodiments of the methods and compounds of the invention, the constituent variables of Formulas (I) and (VII) are selected from those of Examples 1-82. Additionally, because of the excellent activity of each of these compounds, each of these compounds is individually preferred and is also preferred as a member of a group that includes any or all of the compounds of Examples 1-82, and in the methods described herein. Each of these compounds also are preferred for use in preparation of medicaments for treating biological conditions.

However, as compounds that cause HCV inhibition at higher concentrations, such as 10 μM, 20 μM or 50 μM in the assays described herein, can still be useful, the present invention is not intended to be limited to compounds having activity of 10 μM or less.

It is intended that each of the patents, applications, and printed publications including books mentioned in this patent document be hereby incorporated by reference in their entirety.

As those skilled in the art will appreciate, numerous changes and modifications may be made to the preferred embodiments of the invention without departing from the spirit of the invention. It is intended that all such variations fall within the scope of the invention.

What is claimed is:

1. A compound of Formula I: 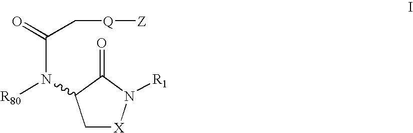
or a stereoisomer or pharmaceutically acceptable salt thereof, wherein:
Q is O, S, SO, SO₂ or N(R₂₅);
R₂₅ is H or alkyl;
R₈₀ is alkyl optionally substituted with up to three independently selected R₆₀ groups, or arylalkyl optionally substituted with up to three independently selected R₃ groups;
each R₃ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl, alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl;
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
or two R₃ groups, when located on adjacent carbon atoms, together can form a moiety of Formula —(O)ₐ—(CH₂)_{b}—(O)_{c}—(CH₂)_{d}—(O)ₑ— wherein a, c and e are independently 0 or 1, and b and d are independently 0, 1, 2 or 3; provided that said moiety does not contain two adjacent oxygen atoms, and that the sum of a, b, c, d and e is at least 3;
R₁ is selected from the group consisting of H, alkyl, alkenyl, alkynyl, aryl, arylalkyl, heteroaryl and heteroarylalkyl, wherein said alkyl is optionally substituted with up to three independently selected R₆₀ groups, and said alkeny, alkynyl, aryl, arylalkyl heteroaryl and heteroarylalkyl are each optionally substituted with up to three independently selected R₆₁ groups;
each R₆₁ is independently selected from the group consisting of OH, C₁₋₆ alkoxy, C₁₋₆ hydroxyalkyl, C₂₋₆ alkenyl, C₂₋₆ alkynyl, CN, NO₂, —S—C₁₋₆alkyl, NR₁₂R₁₃, —C(═O)NR₁₂R₁₃, halogen, R₅₀, heteroaryl, heteroarylalkyl, heterocycloalkyl, perhaloalkyl, perhaloalkoxy, amidino, arylalkyloxy, —S-arylalkyl, azido, hydrazino, hydroxylamino, sulfoxy, sulfonyl, sulfide, disulfide, aryl and arylalkyl, wherein said C₁₋₆ alkoxy, C₂₋₆ alkenyl, C₂₋₆ alkynyl, —S—C₁₋₆ alkyl, heteroaryl, heteroarylalkyl, heterocycloalkyl, arylalkyloxy, —S-arylalkyl, aryl and arylalkyl are each optionally substituted with up to three substituents selected from the group consisting of C₁₋₆ alkyl, C₁₋₆ alkoxy, halogen, OH and C₁₋₃ perhaloalkyl;
each R₆₁ is independently selected from the group consisting of R₆₀ and C₁₋₆ alkyl;
X is a single bond, a group of Formula —(CH₂)ₙ— wherein n is 1, 2, 3, 4, or 5; or a group of Formula II: 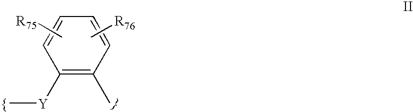
where Y is CH₂, S, SO, SO₂ or N(R₂₀);
R₇₅ and R₇₆ are each independently selected from the group consisting of H, alkyl, alkoxy, alkenyloxy, halogen, aryloxy, heteroaryl, N(R₂₀)(R₂₁) and R₅₀, wherein said alkyloxy, alkenyloxy, aryloxy and heteroaryl are each optionally substituted with up to five independently selected R₆₁ groups, and said alkyl is optionally substituted with up to five independently selected R₆₀ groups;
Z is alkyl, aryl, arylalkyl or heretoaryl, each of which are optionally substituted with up to two independently selected R₂ groups;
each R₂ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy, arylsulfonyl, and a group of Formula —(CH₂)_{f}—N(R₁₁)—(R₁₀);
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
f is 0, 1, 2, 3, 4, 5 or 6;
R₁₁ is H, alkyl or arylalkyl;
R₁₀ is alkyl, alkenyl, cycloalkyl, aryl, arylalkyl, heteroarylalkyl, or cycloalkylalkyl, wherein said arylalkyl, aryl and heteroaryl are each optionally substituted with are each optionally substituted with up to three independently selected R₆₁ groups; and said alkyl is optionally substituted with up to three independently selected R₆₀ groups;
or R₁₁ and R₁₀ together with the nitrogen to which they are attached can form a heterocyclic ring that is optionally substituted with up to three independently selected R₆₁ groups;
R₁₂ and R₁₃ are each independently H, alkyl or arylalkyl;
R₂₀ and R₂₁ are each independently H, alkyl or arylalkyl, wherein said arylalkyl is optionally substituted with up to three independently selected R₆₁ groups, and said alkyl is optionally substituted with up to three independently selected R₆₀ groups; and
R₅₀ is a group of Formula III: 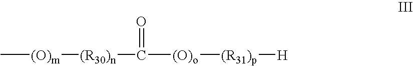
wherein m, n, o and p are each 0 or 1; and
R₃₀ and R₃₁ are each independently C₁₋₆alkyl;
provided that said compound is not N-(4-ethoxybenzyl)-N-(2-oxoazepan-3-yl)-2-phenoxyacetamide or N-[(2-fluorophenyl)methyl]-N-(2-oxoazepan-3-yl)-2,2-diphenylacetamide.

2. The compound, stereoisomer, or pharmaceutically acceptable salt of claim 1 having the Formula IV:

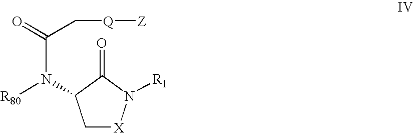

3. The compound, stereoisomer, or pharmaceutically acceptable salt of claim 2 wherein:
R₈₀ is benzyl optionally substituted with up to two independently selected R₃ groups; and
Z has the Formula V or VI: 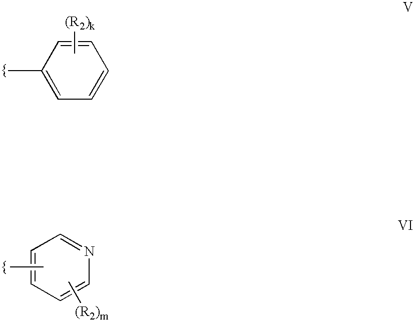
where k and m are each 0, 1 or 2, and each R₂ can be the same or different.

4. The compound, stereoisomer, or pharmaceutically acceptable salt of claim 3 wherein:
each R₃ is independently selected from the group consisting of H, OH, alkyl, alkoxy, alkenyloxy, halogen, aryloxy, heteroaryl, N(R₂₀)(R₂₁), R₅₀, aryl and arylalkyl; wherein said alkoxy, alkenyloxy, aryloxy, heteroaryl, aryl and arylalkyl groups are each optionally substituted with up to five independently selected R₆₁ groups; and said alkyl is optionally substituted with up to five independently selected R₆₀ groups;
or two R₃ groups, when located on adjacent carbon atoms, together can form said moiety of Formula —(O)ₐ—(CH₂)_{b}—(O)_{c}—(CH₂)_{d}—(O)ₑ—; and
each R₂ is independently selected from the group consisting of H, OH, alkyl, alkoxy, alkenyloxy, halogen, aryloxy, heteroaryl, N(R₂₀)(R₂₁), R₅₀, aryl, arylalkyl, and a group of Formula —(CH₂)_{f}—N(R₁₁)—(R₁₀);
wherein said alkoxy, alkenyloxy, aryloxy, heteroaryl, aryl and arylalkyl groups are each optionally substituted with up to five independently selected R₆₁ groups; and said alkyl is optionally substituted with up to five independently selected R₆₀ groups.

5. The compound, stereoisomer or pharmaceutically effective salt of claim 2 wherein:
R₁ is selected from the group consisting of H, benzyl and alkyl;
each R₃ is independently selected from the group consisting of H, OH, C₁₋₆ allyl, C₁₋₆ alkoxy, CF₃, OCF₃, allyloxy, halogen, pyridyl, —C(═O)—OC₁₋₆ alkyl, thiazolyl optionally substituted with a C₁₋₆ alkyl group, phenoxy optionally substituted with up to three substituents selected from the group consisting of halogen, C₁₋₆alkoxy, CF₃ and OCF₃; and N(R₄₀)(R₄₁) where R₄₀ is C₁₋₆ alkyl and R₄₁ is C₁₋₆ alkyl that is optionally substituted with —OC₁₋₆ alkyl;
or two R₃ groups, when located on adjacent carbon atoms, together can form said moiety of Formula —(O)ₐ—(CH₂)_{b}—(O)_{c}—(CH₂)_{d}—(O)ₑ—; and
each R₂ is independently selected from the group consisting of H, OH, C₁₋₆alkyl, C₁₋₆ alkoxy, and a group of Formula —(CH₂)_{f}—N(R₁₁)(R₁₀) wherein:
f is 1;
R₁₁ is H or C₁₋₆ alkyl; and
R₁₀ is an optionally substituted arylalkyl group of Formula —(CH₂)_{g}-L, where g is 0, 1, 2, 3, 4, 5 or 6, and L is selected from the group consisting of H, C₃₋₆ cycloalkyl, allyl, pyridyl and phenyl, wherein said phenyl is optionally substituted with up to three substituents selected from the group consisting of halogen, OH, C₁₋₆ alkyl, OC₁₋₆ alkyl, CF₃, OCF₃ and N(R₁₂)(R₁₃);
or R₁₁ and R₁₀ together with the nitrogen to which they are attached can form piperidine that is optionally substituted with a heterocycloalkyl group.

6. The compound, stereoisomer or pharmaceutically effective salt of any of claims 3, 4 or 5 wherein Z has the Formula V.

7. The compound, stereoisomer or pharmaceutically effective salt of any of claims 3, 4 or 5 wherein Z has the Formula VI.

8. The compound, stereoisomer or pharmaceutically effective salt of any of claims 3, 4 or 5 wherein Q is O.

9. The compound, stereoisomer or pharmaceutically effective salt of any of claims 3, 4 or 5 wherein Q is N(R₂₅).

10. The compound, stereoisomer or pharmaceutically effective salt of any of claims 3, 4 or 5 wherein Q is S.

11. The compound, stereoisomer or pharmaceutically effective salt of any of claims 3, 4 or 5 wherein Q is SO.

12. The compound, stereoisomer or pharmaceutically effective salt of any of claims 3, 4 or 5 wherein Q is SO₂.

13. The compound, stereoisomer or pharmaceutically effective salt of any of claims 3, 4 or 5 wherein X is a group of Formula —(CH₂)ₙ— wherein n is 2 or 3.

14. The compound, stereoisomer or pharmaceutically effective salt of any of claims 3, 4 or 5 wherein X is a group of Formula II wherein Y is CH₂.

15. The compound, stereoisomer or pharmaceutically effective salt of any of claims 3, 4 or 5 wherein X is a group of Formula II wherein Y is S.

16. The compound, stereoisomer or pharmaceutically effective salt of any of claims 3, 4 or 5 wherein X is a group of Formula II wherein Y is SO.

17. The compound, stereoisomer or pharmaceutically effective salt of any of claims 3, 4 or 5 wherein X is a group of Formula II wherein Y is SO₂.

18. The compound, stereoisomer or pharmaceutically effective salt of any of claims 3, 4 or 5 wherein X is a group of Formula II wherein Y is N(R₂₀).

19. The compound, stereoisomer or pharmaceutically effective salt of any of claims 3, 4 or 5 wherein Q is O; Z has the Formula V; and X is a group of Formula —(CH₂)ₙ— wherein n is 2 or 3.

20. The compound, stereoisomer or pharmaceutically effective salt of any of claims 3, 4, or 5 wherein Q is O; Z has the Formula VI; and X is a group of Formula —(CH₂)ₙ— wherein n is 2 or 3.

21. The compound, stereoisomer or pharmaceutically effective salt of any of claims 3, 4 or 5 wherein Q is S; Z has the Formula V; and X is a group of Formula —(CH₂)ₙ— wherein n is 2 or 3.

22. The compound, stereoisomer or pharmaceutically effective salt of any of claims 3, 4, or 5 wherein Q is S; Z has the Formula VI; and X is a group of Formula —(CH₂)ₙ— wherein n is 2 or 3.

23. The compound, stereoisomer or pharmaceutically effective salt of any of claims 3, 4 or 5 wherein Q is O; Z has the Formula V; and X is a group of Formula II.

24. The compound, stereoisomer or pharmaceutically effective salt of claim 23 wherein Y is S.

25. The compound, stereoisomer or pharmaceutically effective salt of any of claims 3, 4, or 5 wherein Q is O; Z has the Formula VI; and X is a group of Formula II.

26. The compound, stereoisomer or pharmaceutically effective salt of claim 25 wherein Y is S.

27. The compound, stereoisomer or pharmaceutically effective salt of any of claims 3, 4 or 5 wherein Q is S; Z has the Formula V; and X is a group of Formula I.

28. The compound, stereoisomer or pharmaceutically effective salt of claim 27 wherein Y is S.

29. The compound, stereoisomer or pharmaceutically effective salt of any of claims 3, 4, or 5 wherein Q is S; Z has the Formula VI; and X is a group of Formula II.

30. The compound, stereoisomer or pharmaceutically effective salt of claim 29 wherein Y is S.

31. The compound, stereoisomer or pharmaceutically effective salt of claim 2 that is selected from the group consisting of:
N-[(3S)-1-ethyl-2-oxoazepan-3-yl]-N-(3-methyl-propoxybenzyl)-2-phonoxyacetamide,
N-(3-methyl-4-propoxybenzyl)-N-[(3S)-2-oxoazepan-3-yl]-2-phenoxyacetamide,
N-(4-ethoxy-3-methylbenzyl)-N-[(3S)-2-oxoazepan-3-yl]-2-phenoxyacetamide,
N-[(3S)-1-methyl-2-oxoazepan-3-yl]-N-(3-methyl-4-propoxybenzyl)-2-phenoxyacetamide,
N-(4-isobutylbenzyl)-N-[(3S)-2-oxoazepan-3-yl]-2-phenoxyacetamide,
N-(3-methyl-4-propoxybenzyl)-N-[(3S)-2-oxopiperidin-3-yl]-2-phenoxyacetamide,
2-(4-{[(2-fluorobenzyl)amino]methyl}phenoxy)-N-(3-methyl-4-propoxybenzyl)-N-[(3S)-2-oxoazepan-3-yl]acetamide,
N-(3-methyl-4-propoxybenzyl)-N-[(3R)-4-oxo-2,3,4,5-tetrahydro-1,5-benzothiazepin-3-yl]-2-phenoxyacetamide,
N-[(3S)-2-oxoazepan-3-yl]-2-phenoxy-N-(4-propoxybenzyl)acetamide,
N-[(3S)-2-oxoazepan-3-yl]-2-phenoxy-N-(4-phenoxybenzyl)acetamide,
2-[4-(anilinomethyl)phenoxy]-N-(3-methyl-4-propoxybenzyl)-N-[(3S)-2-oxoazepan-3-yl]acetamide,
2-{4-[(benzylamino)methyl]phenoxy}-N-(3-methyl-4-propoxybenzyl)-N-[(3S)-2-oxoazepan-3-yl]acetamide,
2-4-{[(4-fluorobenzyl)amino]methyl}phenoxy)-N-(3-methyl-4-propoxybenzyl)-N-[(3S)-2-oxoazepan-3-yl]acetamide,
N-[(3S)-1-benzyl-2-oxoazepan-3-yl]-N-(3-methyl-4-propoxybenzyl)-2-phenoxyacetamide,
N-(4-methoxy-3-methylbenzyl)-N-[(3S)-2-oxoazepan-3-yl]-2-phenoxyacetamide,
2-(4-{[(3-fluorobenzyl)amino]methyl}phenoxy)-N-(3-methyl-4-propoxybenzyl)-N-[(3S)-2-oxoazepan-3-yl]acetamide,
N-[3-(allyloxy)benzyl]-N-[(3S)-2-oxoazepan-3-yl]-2-phenoxyacetamide,
N˜1˜-(3-methyl-4-propoxybenzyl)-N˜1˜-[(3S)-2-oxoazepan-3-yl]-N˜2˜-phenylglycinamide,
N-(3-methyl-4-propoxybenzyl)-2-[(6-methylpyridin-3-yl)oxy]-N-[(3S)-2-oxoazepan-3-yl]acetamide,
N-{4-[(2-methoxyethyl)(methyl)amino]benzyl}-N-[(3S)-2-oxoazepan-3-yl]-2-phenoxyacetamide,
N-(4-isopropoxybenzyl)-N-[(3S)-2-oxoazepan-3-yl]-2-phenoxyacetamide,
2-(4-{[(4-methylbenzyl)amino]methyl}phenoxy)-N-(3-methyl-4-propoxybenzyl)-N-[(3S)-2-oxoazepan-3-yl]acetamide,
N-(3-methyl-4-propoxybenzyl)-N-[(3S)-2-oxoazepan-3-yl]-2-(4-{[(2-phenylethyl)amino]methyl}phenoxy)acetamide,
N-(3-methyl-4-propoxybenzyl)-N-[(3S)-2-oxoazepan-3-yl]-2-(phenylthio)acetamide,
N-(4-ethoxybenzyl)-2-(4-fluorophenoxy)-N-[(3S)-2-oxoazepan-3-yl]acetamide,
N-(4-ethoxybenzyl)-N-[(3S)-2-oxoazepan-3-yl]-2-phenoxyacetamide,
N-(3,4-dichlorobenzyl)-N-[(3S)-2-oxoazepan-3-yl]-2-phenoxyacetamide,
2-(4-{[(4-chlorobenzyl)amino]methyl}phenoxy)-N-(3-methyl-4-propoxybenzyl)-N-[(3S)-2-oxoazepan-3-yl]acetamide,
N-(3-methyl-4-propoxybenzyl)-N-[(3S)-2-oxoazepan-3-yl]-2-(4-{[(pyridin-3-ylmethyl)amino]methyl}phenoxy)acetamide,
N-[3-(3,5-dichlorophenoxy)benzyl]-N-[(3S)-2-oxoazepan-3-yl]-2-phenoxyacetamide,
N-(3-methyl-4-propoxybenzyl)-N-[(3S)-2-oxoazepan-3-yl]-2-(4-{[(pyridin-4-ylmethyl)amino]methyl}phenoxy)acetamide,
N-[(3S)-2-oxoazepan-3-yl]-2-phenoxy-N-[4-(trifluoromethyl)benzyl]acetamide,
N-[(3S)-2-oxoazepan-3-yl]-2-phenoxy-N-[4-trifluoromethoxy)benzyl]acetamide,
2-[4-({[4-(dimethylamino)benzyl]amino}methyl)phenoxy]-N-(3-methyl-4-propoxybenzyl)-N-[(3S)-2-oxoazepan-3-yl]acetamide,
N-(4-ethoxybenzyl)-N-(2-oxoazepan-3-yl)-2-phenoxyacetamide,
N-(4-ethoxybenzyl)-N-(2-oxoazepan-3-yl)-2-phenoxyacetamide,
2-[4-({[2-(4-chlorophenyl)ethyl]amino}methyl)phenoxy]-N-(3-methyl-4-propoxybenzyl)-N-[(3S)-2-oxoazepan-3-yl]acetamide,
N-[(3S)-2-oxoazepan-3-yl]-2-phenoxy-N-(4-pyridin-4-ylbenzyl)acetamide,
N-(4-tert-butoxybenzyl)-N-[(3S)-2-oxoazepan-3-yl]-2-phenoxyacetamide,
N-(2,3-dihydro-1,4-benzodioxin-6-ylmethyl)-N-[(3S)-2-oxoazepan-3-yl]-2-phenoxyacetamide,
N-[4-(dimethylamino)benzyl]-N-[(3S)-2-oxoazepan-3-yl]-2-phenoxyacetamide,
2-(4-{[(2,4-dimethoxybenzyl)amino]methyl}phenoxy)-N-(3-methyl-4-propoxybenzyl)-N-[(3S)-2-oxoazepan-3-yl]acetamide,
N-(3-methyl-4-propoxybenzyl)-N-[(3S)-2-oxoazepan-3-yl]-2-(pyridin-2-yloxy)acetamide,
N-[4-(4-tert-butyl-1,3-thiazol-2-yl)benzyl]-N-[(3S)-2-oxoazepan-3-yl]-2-phenoxyacetamide,
2-(4-{[methyl(phenyl)amino]methyl}phenoxy)-N-(3-methyl-4-propoxybenzyl)-N-[(3S)-2-oxoazepan-3-yl]acetamide,
N-[(3S)-2-oxoazepan-3-yl]-2-phenoxy-N-(4-pyridin-2-ylbenzyl)acetamide,
2-(3-chlorophenoxy)-N-(4-ethoxybenzyl)-N-[(3S)-2-oxoazepan-3-yl]acetamide,
N-(2,3-dihydro-1-benzofuran-5-ylmethyl)-N-[(3S)-2-oxoazepan-3-yl]-2-phenoxyacetamide,
N-(3-methyl-4-propoxybenzyl)-N-[(3S)-2-oxoazepan-3-yl]-2-(4-{[(pyridin-2-ylmethyl)amino]methyl}phenoxy)acetamide,
2-[3-(anilinomethyl)phenoxy]-N-(3-methyl-4-propoxybenzyl)-N-[(3S)-2-oxoazepan-3-yl]acetamide,
methyl 4-{[[(3S)-2-oxoazepan-3-yl](phenoxyacetyl)amino]methyl}benzoate,
2-(4-{[methyl(2-phenylethyl)amino]methyl}phenoxy)-N-(3-methyl-4-propoxybenzyl)-N-[(3S)-2-oxoazepan-3-yl]acetamide,
2-[(5-bromopyridin-2-yl)oxy]-N-(3-methyl-4-propoxybenzyl)-N-[(3S)-2-oxoazepan-3-yl]acetamide,
2-[(5-chloropyridin-2-yl)oxy]-N-(3-methyl-4-propoxybenzyl)-N-[(3S)-2-oxoazepan-3-yl]acetamide,
2-[2-(1,4′-bipiperidin-1′-ylmethyl)phenoxy]-N-(3-methyl-4-propoxybenzyl)-N-[(3S)-2-oxoazepan-3-yl]acetamide,
2-[4-(1,4′-bipiperidin-1′-ylmethyl)phenoxy]-N-(3-methyl-4-propoxybenzyl)-N-[(3S)-2-oxoazepan-3-yl]acetamide,
N-[(3S)-2-oxoazepan-3-yl]-2-phenoxy-N-(3-phenoxybenzyl)acetamide,
2-(4-{[(cyclohexylmethyl)amino]methyl}phenoxy)-N-(3-methyl-4-propoxybenzyl)-N-[(3S-2-oxoazepan-3-yl]acetamide,
2-(3-{[methyl(phenyl)amino]methyl}phenoxy)-N-(3-methyl-4-propoxybenzyl)-N-[(3S)-2-oxoazepan-3-yl]acetamide,
N-(3-methyl-4-propoxybenzyl)-N-[(3S)-2-oxoazepan-3-yl]-2-(4-{[(3-phenylpropyl)amino]methyl}phenoxy)acetamide,
N-(3-methyl-4-propoxybenzyl)-N-[(3S)-2-oxoazepan-3-yl]-2-(4-{[(4-phenylbutyl)amino]methyl}phenoxy)acetamide,
2-{4-[(hexylamino)methyl]phenoxy}-N-(3-methyl-4-propoxybenzyl)-N-[(3S)-2-oxoazepan-3-yl]acetamide,
2-3-{[methyl(2-phenylethyl)amino]methyl}phenoxy)-N-(3-methyl-4-propoxybenzyl)-N-[(3S)-2-oxoazepan-3-yl]acetamide,
N-(3-methyl-4-propoxybenzyl)-N-[(3S)-2-oxoazepan-3-yl]-2-(4-{[(2-pyridin-4-ylethyl)amino]methyl}phenoxy)acetamide,
2-(3-{[(cyclohexylmethyl)amino]methyl}phenoxy)-N-(3-methyl-4-propoxybenzyl)-N [(3S)-2-oxoazepan-3-yl]acetamide,
N-(3-methyl-4-propoxybenzyl)-N-[(3S)-2-oxoazepan-3-yl]-2-(3-{((3-phenylpropyl)aminomethyl}phenoxy)acetamide,
2-(3-{[(4-fluorobenzyl)amino]methyl}phenoxy)-N-(3-methyl-4-propoxybenzyl)-N-[(3S)-2-oxoazepan-3-yl]acetamide,
2-[3-({[2-(4-chlorophenyl)ethyl]amino}methyl)phenoxy]-N-(3-methyl-4-propoxybenzyl)-N-[(3S)-2-oxoazepan-3-yl]acetamide,
2-{4-[(allylamino)methyl]phenoxy}-N-(3-methyl-4-propoxybenzyl)-N-[(3S)-2-oxoazepan-3-yl]acetamide,
N-[(3S)-2-oxoazepan-3-yl]-2-phenoxy-N-(4-pyridin-3-ylbenzyl)acetamide,
N-(3-bromobenzyl)-N-[(3S)-2-oxoazepan-3-yl]-2-phenoxyacetamide,
N-(3-methyl-4-propoxybenzyl)-N-[(3S)-2-oxoazepan-3-yl]-2-(pyridin-4-yloxy)acetamide,
N-(4-methoxybenzyl)-N-[(3S)-2-oxoazepan-3-yl]-2-phenoxyacetamide,
N-(3,4-dimethoxybenzyl)-N-[(3S)-2-oxoazepan-3-yl]-2-phenoxyacetamide, and
2-[(2-iodopyridin-3-yl)oxy]-N-(3-methyl-4-propoxybenzyl)-N-[(3S)-2-oxoazepan-3-yl]acetamide.

32. A composition comprising a compound, stereoisomer or pharmaceutically effective salt of any of claims 1-5.

33. A pharmaceutical composition comprising a compound, stereoisomer or pharmaceutically effective salt of any of claims 1-5.

34. A method for treating a viral infection comprising administering to a patient suffering from said infection a compound of Formula I: 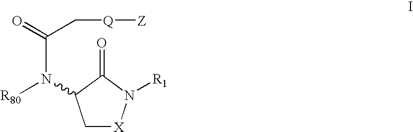
or a stereoisomer or pharmaceutically acceptable salt thereof, wherein:
Q is O, S, SO, SO₂ or N(R₂₅);
R₂₅ is H or alkyl;
R₈₀ is alkyl optionally substituted with up to three independently selected R₆₀ groups, or arylalkyl optionally substituted with up to three independently selected R₃ groups;
each R₃ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-allyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl;
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
or two R₃ groups, when located on adjacent carbon atoms, together can form a moiety of Formula —(O)ₐ—(CH₂)_{b}—(O)_{c}—(CH₂)_{d}—(O)ₑ— wherein a, c and e are independently 0 or 1, and b and d are independently 0, 1, 2 or 3; provided that said moiety does not contain two adjacent oxygen atoms, and that the sum of a, b, c, d and e is at least 3;
R₁ is selected from the group consisting of H, alkyl, alkenyl, alkynyl, aryl, arylalkyl, heteroaryl and heteroarylalkyl, wherein said alkyl is optionally substituted with up to three independently selected R₆₀ groups, and said alkeny, alkynyl, aryl, arylalkyl heteroaryl and heteroarylalkyl are each optionally substituted with up to three independently selected R₆₁ groups;
each R₆₀ is independently selected from the group consisting of OH, C₁₋₆ alkoxy, C₁₋₆ hydroxyalkyl, C₂₋₆ alkenyl, C₂₋₆ alkynyl, CN, NO₂, —S—C₁₋₆ alkyl, NR₁₂R₁₃, —C(═O)NR₁₂R₁₃, halogen, R₅₀, heteroaryl, heteroarylalkyl, heterocycloalkyl, perhaloalkyl, perhaloalkoxy, amidino, arylalkyloxy, —S-arylalkyl, azido, hydrazino, hydroxylamino, sulfoxy, sulfonyl, sulfide, disulfide, aryl and arylalkyl, wherein said C₁₋₆ alkoxy, C₂₋₆ alkenyl, C₂₋₆ alkynyl, —S—C₁₋₆ alkyl, heteroaryl, heteroarylalkyl, heterocycloalkyl, arylalkyloxy, —S-arylalkyl, aryl and arylalkyl are each optionally substituted with up to three substituents selected from the group consisting of C₁₋₆ alkyl, C₁₋₆ alkoxy, halogen, OH and C₁₋₃ perhaloalkyl;
each R₆₁ is independently selected from the group consisting of R₆₀ and C₁₋₆ alkyl;
X is a single bond, a group of Formula —(CH₂)ₙ— wherein n is 1, 2, 3, 4, or 5; or a group of Formula II: 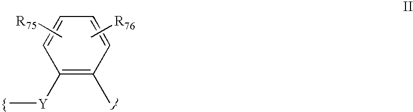
where Y is CH₂, S, SO, SO₂ or N(R₂₀);
R₇₅ and R₇₆ are each independently selected from the group consisting of H, alkyl, alkoxy, alkenyloxy, halogen, aryloxy, heteroaryl, N(R₂₀)(R₂₁) and R₅₀, wherein said alkyloxy, alkenyloxy, aryloxy and heteroaryl are each optionally substituted with up to five independently selected R₆₁ groups, and said alkyl is optionally substituted with up to five independently selected R₆₀ groups;
Z is alkyl, aryl, arylalkyl or heretoaryl, each of which are optionally substituted with up to two independently selected R₂ groups;
each R₂ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O) arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy, arylsulfonyl, and a group of Formula —(CH₂)_{f}—N(R₁₁)—(R₁₀);
wherein said alkoxy, alkenyloxy, aryloxy, heteroaryl, alkylsulfonyl, S-allyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
f is 0, 1, 2, 3, 4, 5 or 6;
R₁₁ is H, alkyl or arylalkyl;
R₁₀ is alkyl, alkenyl, cycloalkyl, aryl, arylalkyl, heteroarylalkyl, or cycloalkylalkyl, wherein said arylalkyl, aryl and heteroaryl are each optionally substituted with are each optionally substituted with up to three independently selected R₆₁ groups; and said alkyl is optionally substituted with up to three independently selected R₆₀ groups;
or R₁₁ and R₁₀ together with the nitrogen to which they are attached can form a heterocyclic ring that is optionally substituted with up to three independently selected R₆₁ groups;
R₁₂ and R₁₃ are each independently H, alkyl or arylalkyl;
R₂₀ and R₂₁ are each independently H, alkyl or arylalkyl, wherein said arylalkyl is optionally substituted with up to three independently selected R₆₁ groups, and said alkyl is optionally substituted with up to three independently selected R₆₀ groups; and
R₅₀ is a group of Formula III: 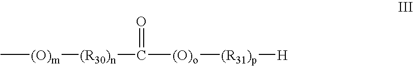
wherein m, n, o and p are each 0 or 1; and
R₃₀ and R₃₁ are each independently C₁₋₆ alkyl.

35. A method for treating a viral infection comprising administering to a patient suffering from said infection a compound, stereoisomer or pharmaceutically effective salt of any of claims 1-5.

36. A method for alleviating a symptom of a viral infection comprising administering to a patient suffering from said infection a compound of Formula I: 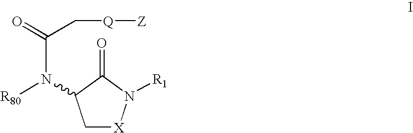
or a stereoisomer or pharmaceutically acceptable salt thereof, wherein:
Q is O, S, SO, SO₂ or N(R₂₅);
R₂₅ is H or alkyl;
R₈₀ is alkyl optionally substituted with up to three independently selected R₆₀ groups, or arylalkyl optionally substituted with up to three independently selected R₃ groups;
each R₃ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl;
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
or two R₃ groups, when located on adjacent carbon atoms, together can form a moiety of Formula —(O)ₐ—(CH₂)_{b}—(O)_{c}—(CH₂)_{d}—(O)ₑ— wherein a, c and e are independently 0 or 1, and b and d are independently 0, 1, 2 or 3; provided that said moiety does not contain two adjacent oxygen atoms, and that the sum of a, b, c, d and e is at least 3;
R₁ is selected from the group consisting of H, alkyl, alkenyl, alkynyl, aryl, arylalkyl, heteroaryl and heteroarylalkyl, wherein said alkyl is optionally substituted with up to three independently selected R₆₀ groups, and said alkeny, alkynyl, aryl, arylalkyl heteroaryl and heteroarylalkyl are each optionally substituted with up to three independently selected R₆₁ groups;
each R₆₀ is independently selected from the group consisting of OH, C₁₋₆ alkoxy, C₁₋₆ hydroxyalkyl, C₂₋₆ alkenyl, C₂₋₆ alkynyl, CN, NO₂, —S—C₁₋₆ alkyl, NR₁₂R₁₃, —C(═O)NR₁₂R₁₃, halogen, R₅₀, heteroaryl, heteroarylalkyl, heterocycloalkyl, perhaloalkyl, perhaloalkoxy, amidino, arylalkyloxy, —S-arylalkyl, azido, hydrazino, hydroxylamino, sulfoxy, sulfonyl, sulfide, disulfide, aryl and arylalkyl, wherein said C₁₋₆ alkoxy, C₂₋₆ alkenyl, C₂₋₆ alkynyl, —S—C₁₋₆ alkyl, heteroaryl, heteroarylalkyl, heterocycloalkyl, arylalkyloxy, —S-arylalkyl, aryl and arylalkyl are each optionally substituted with up to three substituents selected from the group consisting of C₁₋₆ alkyl, C₁₋₆ alkoxy, halogen, OH and C₁₋₃ perhaloalkyl;
each R₆₁ is independently selected from the group consisting of R₆₀ and C₁₋₆ alkyl;
X is a single bond, a group of Formula (CH₂)ₙ— wherein n is 1, 2, 3, 4, or 5; or a group of Formula II: 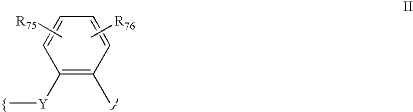
where Y is CH₂, S, SO, SO₂ or N(R₂₀);
R₇₅ and R₇₆ are each independently selected from the group consisting of H, alkyl, alkoxy, alkenyloxy, halogen, aryloxy, heteroaryl, N(R₂₀)(R₂₁) and R₅₀, wherein said alkyloxy, alkenyloxy, aryloxy and heteroaryl are each optionally substituted with up to five independently selected R₆₁ groups, and said alkyl is optionally substituted with up to five independently selected R₆₀ groups;
Z is alkyl, aryl, arylalkyl or heretoaryl, each of which are optionally substituted with up to two independently selected R₂ groups;
each R₂ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy, arylsulfonyl, and a group of Formula —(CH₂)_{f}—N(R₁₁)—(R₁₀);
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
f is 0, 1, 2, 3, 4, 5 or 6;
R₁₁ is H, alkyl or arylalkyl;
R₁₀ is alkyl, alkenyl, cycloalkyl, aryl, arylalkyl, heteroarylalkyl, or cycloalkylalkyl, wherein said arylalkyl, aryl and heteroaryl are each optionally substituted with are each optionally substituted with up to three independently selected R₆₁ groups; and said alkyl is optionally substituted with up to three independently selected R₆₀ groups;
or R₁₁ and R₁₀ together with the nitrogen to which they are attached can form a heterocyclic ring that is optionally substituted with up to three independently selected, 61 groups;
R₁₂ and R₁₃ are each independently H, alkyl or arylalkyl;
R₂₀ and R₂₁ are each independently H, alkyl or arylalkyl, wherein said arylalkyl is optionally substituted with up to three independently selected R₆₁ groups, and said alkyl is optionally substituted with up to three independently selected R₆₀ groups; and
R₅₀ is a group of Formula III: 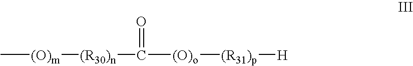
wherein m, n, o and p are each 0 or 1; and
R₃₀ and R₃₁ are each independently C₁₋₆ alkyl.

37. A method for alleviating a symptom of a viral infection comprising administering to a patient suffering from said infection a pharmaceutical composition comprising A compound of Formula I: 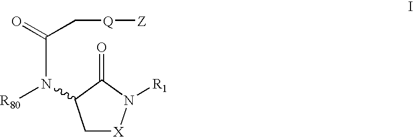
or a stereoisomer or pharmaceutically acceptable salt thereof, wherein:
Q is O, S, SO, SO₂ or N(R₂₅);
R₂₅ is H or alkyl;
R₈₀ is alkyl optionally substituted with up to three independently selected R₆₀ groups, or arylalkyl optionally substituted with up to three independently selected R₃ groups;
each R₃ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl;
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
or two R₃ groups, when located on adjacent carbon atoms, together can form a moiety of Formula —(O)ₐ—(CH₂)_{b}—(O)_{c}—(CH₂)_{d}—(O)ₑ— wherein a, c and e are independently 0 or 1, and b and d are independently 0, 1, 2 or 3; provided that said moiety does not contain two adjacent oxygen atoms, and that the sum of a, b, c, d and e is at least 3;
R₁ is selected from the group consisting of H, alkyl, alkenyl, alkynyl, aryl, arylalkyl, heteroaryl and heteroarylalkyl, wherein said alkyl is optionally substituted with up to three independently selected R₆₀ groups, and said alkeny, alkynyl, aryl, arylalkyl heteroaryl and heteroarylalkyl are each optionally substituted with up to three independently selected R₆₁ groups;
each ho is independently selected from the group consisting of OH, C₁₋₆ alkoxy, C₁₋₆ hydroxyalkyl, C₂₋₆ alkenyl, C₂₋₆ alkynyl, CN, NO₂, —S—C₁₋₆ alkyl, NR₁₂R₁₃, —C(═O)NR₁₂R₁₃, halogen, R₅₀, heteroaryl, heteroarylalkyl, heterocycloalkyl, perhaloalkyl, perhaloalkoxy, amidino, arylalkyloxy, —S-arylalkyl, azido, hydrazino, hydroxylamino, sulfoxy, sulfonyl, sulfide, disulfide, aryl and arylalkyl, wherein said C₁₋₆ alkoxy, C₂₋₆ alkenyl, C₂₋₆ alkynyl, —S—C₁₋₆ alkyl, heteroaryl, heteroarylalkyl, heterocycloalkyl, arylalkyloxy, —S-arylalkyl, aryl and arylalkyl are each optionally substituted with up to three substituents selected from the group consisting of C₁₋₆ alkyl, C₁₋₆ alkoxy, halogen, OH and C₁₋₃ perhaloalkyl;
each R₆₁ is independently selected from the group consisting of R₆₀ and C₁₋₆ alkyl;
X is a single bond, a group of Formula —(CH₂)ₙ— wherein n is 1, 2, 3, 4, or 5; or a group of Formula II: 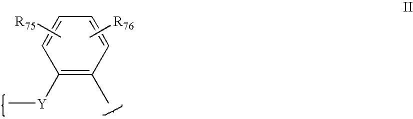
where Y is CH₂, S, SO, SO₂ or N(R₂₀);
R₇₅ and R₇₆ are each independently selected from the group consisting of H, alkyl, alkoxy, alkenyloxy, halogen, aryloxy, heteroaryl, N(R₂₀)(R₂₁) and R₅₀, wherein said alkyloxy, alkenyloxy, aryloxy and heteroaryl are each optionally substituted with up to five independently selected R₆₁ groups, and said alkyl is optionally substituted with up to five independently selected R₆₀ groups;
Z is alkyl, aryl, arylalkyl or heretoaryl, each of which are optionally substituted with up to two independently selected R₂ groups;
each R₂ is independently selected from the group consisting of H, OH, allyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy, arylsulfonyl, and a group of Formula —(CH₂)_{f}—N(R₁₁)—(R₁₀);
wherein said alkoxy, alkenyloxy, aryloxy, heteroaryl, alkylsulfonyl, S-allyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
f is 0, 1, 2, 3, 4, 5 or 6;
R₁₁ is H, alkyl or arylalkyl;
R₁₀ is alkyl, alkenyl, cycloalkyl, aryl, arylalkyl, heteroarylalkyl, or cycloalkylalkyl, wherein said arylalkyl, aryl and heteroaryl are each optionally substituted with are each optionally substituted with up to three independently selected R₆₁ groups; and said alkyl is optionally substituted with up to three independently selected R₆₀ groups;
or R₁₁ and R₁₀ together with the nitrogen to which they are attached can form a heterocyclic ring that is optionally substituted with up to three independently selected R₆ groups;
R₁₂ and R₁₃ are each independently H, alkyl or arylalkyl;
R₂₀ and R₂₁ are each independently H, alkyl or arylalkyl, wherein said arylalkyl is optionally substituted with up to three independently selected R₆₁ groups, and said alkyl is optionally substituted with up to three independently selected R₆₀ groups; and
R₅₀ is a group of Formula III: 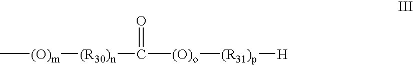
wherein m, n, o and p are each 0 or 1; and
R₃₀ and R₃₁ are each independently C₁₋₆ alkyl.

38. A method for alleviating a symptom of HCV comprising administering to a patient suffering from said infection a compound of Formula I: 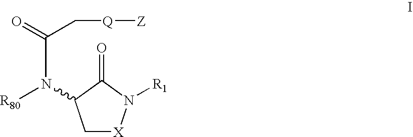
or a stereoisomer or pharmaceutically acceptable salt thereof, wherein:
Q is O, S, SO, SO₂ or N(R₂₅);
R₂₅ is H or alkyl;
R₈₀ is alkyl optionally substituted with up to three independently selected R₆₀ groups, or arylalkyl optionally substituted with up to three independently selected R₃ groups;
each R₃ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C((═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl;
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
or two R₃ groups, when located on adjacent carbon atoms, together can form a moiety of Formula —(O)ₐ—(CH₂)_{b}—(O)_{c}—(CH₂)_{d}—(O)ₑ— wherein a, c and e are independently 0 or 1, and b and d are independently 0, 1, 2 or 3; provided that said moiety does not contain two adjacent oxygen atoms, and that the sum of a, b, c, d and e is at least 3;
R₁ is selected from the group consisting of H, alkyl, alkenyl, alkynyl, amyl, arylalkyl, heteroaryl and heteroarylalkyl, wherein said alkyl is optionally substituted with up to three independently selected R₆₀ groups, and said alkeny, alkynyl, aryl, arylalkyl heteroaryl and heteroarylalkyl are each optionally substituted with up to three independently selected R₆₁ groups;
each R₅₀ is independently selected from the group consisting of OH, C₁₋₆ alkoxy, C₁₋₆ hydroxyalkyl, C₂₋₆ alkenyl, C₂₋₆ alkynyl, CN, NO₂, —S—C₁₋₆ alkyl, NR₁₂R₁₃, —C(═O)NR₁₂R₁₃, halogen, R₅₀, heteroaryl, heteroarylalkyl, heterocycloalkyl, perhaloalkyl, perhaloalkoxy, amidino, arylalkyloxy, —S-arylalkyl, azido, hydrazino, hydroxylamino, sulfoxy, sulfonyl, sulfide, disulfide, aryl and arylalkyl, wherein said C₁₋₆ alkoxy, C₂₋₆ alkenyl, C₂₋₆ alkynyl, —S—CO₁ alkyl, heteroaryl, heteroarylalkyl, heterocycloalkyl, arylalkyloxy, —S-arylalkyl, aryl and arylalkyl are each optionally substituted with up to three substituents selected from the group consisting of C₁₋₆ alkyl, C₁₋₆ alkoxy, halogen, OH and C₁₋₃ perhaloalkyl;
each R₆₋₁ is independently selected from the group consisting of R₆₀ and C₁₋₆ alkyl;
X is a single bond, a group of Formula —(CH₂)ₙ— wherein n is 1, 2, 3, 4, or 5; or a group of Formula II: 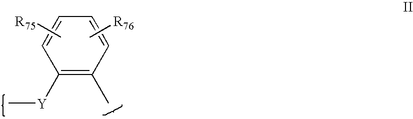
where Y is CH₂, S, SO, SO₂ or N(R₂₀);
R₇₅ and R₇₆ are each independently selected from the group consisting of H, alkyl, alkoxy, alkenyloxy, halogen, aryloxy, heteroaryl, N(R₂₀)(R₂₁) and R₅₀, wherein said alkyloxy, alkenyloxy, aryloxy and heteroaryl are each optionally substituted with up to five independently selected R₆₁ groups, and said alkyl is optionally substituted with up to five independently selected R₆₀ groups;
Z is alkyl, aryl, arylalkyl or heretoaryl, each of which are optionally substituted with up to two independently selected R₂ groups;
each R₂ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy, arylsulfonyl, and a group of Formula —(CH₂)_{f}—N(R₁₁)—(R₁₀);
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
f is 0, 1, 2, 3, 4, 5 or 6;
R₁₁ is H, alkyl or arylalkyl;
R₁₀ is alkyl, alkenyl, cycloalkyl, aryl, arylalkyl, heteroarylalkyl, or cycloalkylalkyl, wherein said arylalkyl, aryl and heteroaryl are each optionally substituted with are each optionally substituted with up to three independently selected R₆₁ groups; and said alkyl is optionally substituted with up to three independently selected R₆₀ groups;
or R₁₁ and R₁₀ together with the nitrogen to which they are attached can form a heterocyclic ring that is optionally substituted with up to three independently selected R₆₁ groups;
R₁₂ and R₁₃ are each independently H, alkyl or arylalkyl;
R₂₀ and R₂₁ are each independently H, alkyl or arylalkyl, wherein said arylalkyl is optionally substituted with up to three independently selected R₆₁ groups, and said alkyl is optionally substituted with up to three independently selected R₆₀ groups; and
R₅₀ is a group of Formula III: 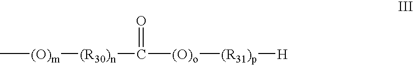
wherein m, n, o and p are each 0 or 1; and
R₃₀ and R₃₁ are each independently C₁₋₆ alkyl.

39. A method for alleviating a symptom of HCV comprising administering to a patient suffering from said infection a pharmaceutical composition comprising a compound of Formula I: 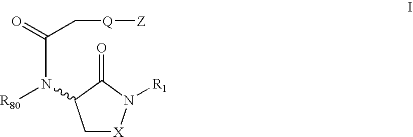
or a stereoisomer or pharmaceutically acceptable salt thereof, wherein:
Q is O, S, SO, SO₂ or N(R₂₅);
R₂₅ is H or alkyl;
R₈₀ is alkyl optionally substituted with up to three independently selected R₆₀ groups, or arylalkyl optionally substituted with up to three independently selected R₃ groups;
each R₃ is independently selected from the group consisting of H, OH, alkyl, alkoxy, alkenyloxy, halogen, aryloxy, heteroaryl, N(R₂₀)R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl;
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
or two R₃ groups, when located on adjacent carbon atoms, together can form a moiety of Formula —(O)ₐ—(CH₂)_{b}—(O)_{c}—(CH₂)_{d}—(O)ₑ— wherein a, c and e are independently 0 or 1, and b and d are independently 0, 1, 2 or 3; provided that said moiety does not contain two adjacent oxygen atoms, and that the sum of a, b, c, d and e is at least 3;
R₁ is selected from the group consisting of H, alkyl, alkenyl, alkynyl, aryl, arylalkyl, heteroaryl and heteroarylalkyl, wherein said alkyl is optionally substituted with up to three independently selected R₆₀ groups, and said alkeny, alkynyl, aryl, arylalkyl heteroaryl and heteroarylalkyl are each optionally substituted with up to three independently selected R₆₁ groups;
each R₆₀ is independently selected from the group consisting of OH, C₁₋₆ alkoxy, C₁₋₆ hydroxyalkyl, C₂₋₆ alkenyl, C₂₋₆ alkynyl, CN, NO₂, —S—C₁₋₆ alkyl, NR₁₂R₁₃, —C(═O)NR₁₂R₁₃, halogen, R₅₀, heteroaryl, heteroarylalkyl, heterocycloalkyl, perhaloalkyl, perhaloalkoxy, amidino, arylalkyloxy, —S-arylalkyl, azido, hydrazino, hydroxylamino, sulfoxy, sulfonyl, sulfide, disulfide, aryl and arylalkyl, wherein said C₁₋₆ alkoxy, C₂₋₆ alkenyl, C₂₋₆ alkynyl, —S—C₁₋₆ alkyl, heteroaryl, heteroarylalkyl, heterocycloalkyl, arylalkyloxy, —S-arylalkyl, aryl and arylalkyl are each optionally substituted with up to three substituents selected from the group consisting of C₁₋₆ alkyl, C₁₋₆ alkoxy, halogen, OH and C₁₋₃ perhaloalkyl;
each R₆₁ is independently selected from the group consisting of R₆₀ and C₁₋₆alkyl;
X is a single bond, a group of Formula —(CH₂)ₙ— wherein n is 1, 2, 3, 4, or 5; or a group of Formula II: 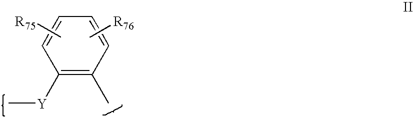
where Y is CH₂, S, SO, SO₂ or N(R₂₀);
R₇₅ and R₇₆ are each independently selected from the group consisting of H, alkyl, alkoxy, alkenyloxy, halogen, aryloxy, heteroaryl, N(R₂₀)(R₂₁) and R₅₀, wherein said alkyloxy, alkenyloxy, aryloxy and heteroaryl are each optionally substituted with up to five independently selected R₆₁ groups, and said alkyl is optionally substituted with up to five independently selected R₆₀ groups;
Z is alkyl, aryl, arylalkyl or heretoaryl, each of which are optionally substituted with up to two independently selected R₂ groups;
each R₂ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, C(═O)arylalkyloxy, arylalkanoylalkyl, heteroaryl, heteroarylalkyl, alkylheteroaryl, heteroarylalkylamino, heteroarylalkylaminoalkyl, arylalkyloxy, arylsulfonyl, and a group of Formula —(CH₂)_{f}—N(R₁₁)—(R₁₀);
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
f is 0, 1, 2, 3, 4, 5 or 6;
R₁₁ is H, alkyl or arylalkyl;
R₁₀ is alkyl, alkenyl, cycloalkyl, aryl, arylalkyl, heteroarylalkyl, or cycloalkylalkyl, wherein said arylalkyl, aryl and heteroaryl are each optionally substituted with are each optionally substituted with up to three independently selected R₆₁ groups; and said alkyl is optionally substituted with up to three independently selected R₆₀ groups;
or R₁₁ and R₁₀ together with the nitrogen to which they are attached can form a heterocyclic ring that is optionally substituted with up to three independently selected R₆₁ groups;
R₁₂ and R₁₃ are each independently H, alkyl or arylalkyl;
R₂₀ and R₂₁ are each independently H, alkyl or arylalkyl, wherein said arylalkyl is optionally substituted with up to three independently selected R₆₁ groups, and said alkyl is optionally substituted with up to three independently selected R₆₀ groups; and
R₅₀ is a group of Formula III: 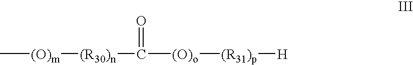
wherein m, n, o and p are each 0 or 1; and
R₃₀ and R₃₁ are each independently Clue alkyl.

40. A method for alleviating a symptom of SARS comprising administering to a patient suffering from said infection a compound of Formula I: 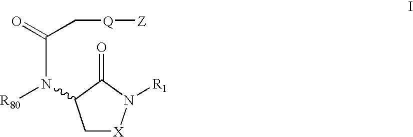
or a stereoisomer or pharmaceutically acceptable salt thereof, wherein:
Q is O, S, SO, SO₂ or N(R₂₅);
R₂₅ is H or alkyl;
R₈₀ is alkyl optionally substituted with up to three independently selected R₆₀ groups, or arylalkyl optionally substituted with up to three independently selected R₃ groups;
each R₃ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl;
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
or two R₃ groups, when located on adjacent carbon atoms, together can form a moiety of Formula —(O)ₐ—(CH₂)_{b}(O)_{c}—(CH₂)_{d}—(O)ₑ— wherein a, c and e are independently 0 or 1, and b and d are independently 0, 1, 2 or 3; provided that said moiety does not contain two adjacent oxygen atoms, and that the sum of a, b, c, d and e is at least 3;
R₁ is selected from the group consisting of H, alkyl, alkenyl, alkynyl, aryl, arylalkyl, heteroaryl and heteroarylalkyl, wherein said alkyl is optionally substituted with up to three independently selected R₄₀ groups, and said alkeny, alkynyl, aryl, arylalkyl heteroaryl and heteroarylalkyl are each optionally substituted with up to three independently selected R₆₁ groups;
each R₆₀ is independently selected from the group consisting of OH, C₁₋₆ alkoxy, C₁₋₆ hydroxyalkyl, C₂₋₆ alkenyl, C₂₋₆ alkynyl, CN, NO₂, —S—C₁₋₆ alkyl, NR₁₂R₁₃, —C(═O)NR₁₂R₁₃, halogen, R₅₀, heteroaryl, heteroarylalkyl, heterocycloalkyl, perhaloalkyl, perhaloalkoxy, amidino, arylalkyloxy, —S-arylalkyl, azido, hydrazino, hydroxylamino, sulfoxy, sulfonyl, sulfide, disulfide, aryl and arylalkyl, wherein said C₁₋₆ alkoxy, C₂₋₆ alkenyl, C₂₋₆ alkynyl, —S—C₁₋₆ alkyl, heteroaryl, heteroarylalkyl, heterocycloalkyl, arylalkyloxy, —S-arylalkyl, aryl and arylalkyl are each optionally substituted with up to three substituents selected from the group consisting of C₁₋₆ alkyl, C₁₋₆ alkoxy, halogen, OH and C₁₋₃ perhaloalkyl;
each R₆₁ is independently selected from the group consisting of R₆₀ and C₁₋₆ alkyl;
X is a single bond, a group of Formula —(CH₂)ₙ— wherein n is 1, 2, 3, 4, or 5; or a group of Formula II: 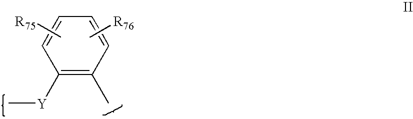
where Y is CH₂, S, SO, SO₂ or N(R₂₀);
R₇₅ and R₇₆ are each independently selected from the group consisting of H, alkyl, alkoxy, alkenyloxy, halogen, aryloxy, heteroaryl, N(R₂₀)(R₂₁) and R₅₀, wherein said alkyloxy, alkenyloxy, aryloxy and heteroaryl are each optionally substituted with up to five independently selected R₆₁ groups, and said alkyl is optionally substituted with up to five independently selected R₆₀ groups;
Z is alkyl, aryl, arylalkyl or heretoaryl, each of which are optionally substituted with up to two independently selected R₂ groups;
each R₂ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy, arylsulfonyl, and a group of Formula —(CH₂)_{f}—N(R₁₁)—(R₁₀);
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
f is 0, 1, 2, 3, 4, 5 or 6;
R₁₁ is H, alkyl or arylalkyl;
R₁₀ is alkyl, alkenyl, cycloalkyl, aryl, arylalkyl, heteroarylalkyl, or cycloalkylalkyl, wherein said arylalkyl, aryl and heteroaryl are each optionally substituted with are each optionally substituted with up to three independently selected R₆₁ groups; and said alkyl is optionally substituted with up to three independently selected R₆₀ groups;
or R₁₁ and R₁₀ together with the nitrogen to which they are attached can form a heterocyclic ring that is optionally substituted with up to three independently selected R₆₁ groups;
R₁₂ and R₁₃ are each independently H, alkyl or arylalkyl;
R₂₀ and R₂₁ are each independently H, alkyl or arylalkyl, wherein said arylalkyl is optionally substituted with up to three independently selected R₆₁ groups, and said alkyl is optionally substituted with up to three independently selected R₆₀ groups; and
R₅₀ is a group of Formula III: 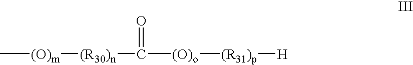
wherein m, n, o and p are each 0 or 1; and
R₃₀ and R₃₁ are each independently C₁₋₆ alkyl.

41. A method for alleviating a symptom of SARS comprising administering to a patient suffering from said infection a pharmaceutical composition comprising a compound of Formula I: 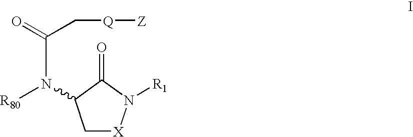
or a stereoisomer or pharmaceutically acceptable salt thereof, wherein:
Q is O, S, SO, SO₂ or N(R₂₅);
R₂₅ is H or alkyl;
R₈₀ is alkyl optionally substituted with up to three independently selected R₆₀ groups, or arylalkyl optionally substituted with up to three independently selected R₃ groups;
each R₃ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl;
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
or two R₃ groups, when located on adjacent carbon atoms, together can form a moiety of Formula —(O)ₐ—(CH₂)_{b}—(O)_{c}—(CH₂)_{d}—(O)ₑ— wherein a, c and e are independently 0 or 1, and b and d are independently 0, 1, 2 or 3; provided that said moiety does not contain two adjacent oxygen atoms, and that the sum of a, b, c, d and e is at least 3;
R₁ is selected from the group consisting of H, alkyl, alkenyl, alkynyl, aryl, arylalkyl, heteroaryl and heteroarylalkyl, wherein said alkyl is optionally substituted with up to three independently selected R₆₀ groups, and said alkeny, alkynyl, aryl, arylalkyl heteroaryl and heteroarylalkyl are each optionally substituted with up to three independently selected R₆₁ groups;
each R₆₀ is independently selected from the group consisting of OH, C₁₋₆ alkoxy, C₁₋₆ hydroxyalkyl, C₂₋₆ alkenyl, C₂₋₆ alkynyl, CN, NO₂, —S—C₁₋₆ alkyl, NR₁₂R₁₃, —C(═O)NR₁₂R₁₃, halogen, R₅₀, heteroaryl, heteroarylalkyl, heterocycloalkyl, perhaloalkyl, perhaloalkoxy, amidino, arylalkyloxy, —S-arylalkyl, azido, hydrazino, hydroxylamino, sulfoxy, sulfonyl, sulfide, disulfide, aryl and arylalkyl, wherein said C₁₋₆ alkoxy, C₂₋₆ alkenyl, C₂₋₆ alkynyl, —S—C₁₋₆ alkyl, heteroaryl, heteroarylalkyl, heterocycloalkyl, arylalkyloxy, —S-arylalkyl, aryl and arylalkyl are each optionally substituted with up to three substituents selected from the group consisting of C₁₋₆alkyl, C₁₋₆ alkoxy, halogen, OH and C₁₋₃ perhaloalkyl;
each R₆₁ is independently selected from the group consisting of R₆₀ and C₁₋₆ alkyl;
X is a single bond, a group of Formula —(CH₂)ₙ— wherein n is 1, 2, 3, 4, or 5; or a group of Formula II: 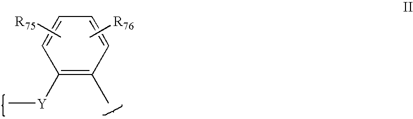
where Y is CH₂, S, SO, SO₂ or N(R₂₀);
R₇₅ and R₇₆ are each independently selected from the group consisting of H, alkyl, alkoxy, alkenyloxy, halogen, aryloxy, heteroaryl, N(R₂₀)(R₂₁) and R₅₀, wherein said alkyloxy, alkenyloxy, aryloxy and heteroaryl are each optionally substituted with up to five independently selected R₆₁ groups, and said alkyl is optionally substituted with up to five independently selected R₆₀ groups;
Z is alkyl, aryl, arylalkyl or heretoaryl, each of which are optionally substituted with up to two independently selected R₂ groups;
each R₂ is independently selected from the group consisting of H, OH, alkyl, alkoxy, alkenyloxy, halogen, aryloxy, heteroaryl, N(R₂₀)(R₂₁), R₅₀, carbamoyl, carbamoylamino, carbamoyloxy, NO₂, azido, hydrazino, hydroxylamino, sulfoxy, sulfonyl, sulfide, disulfide, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═C)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy, arylsulfonyl, and a group of Formula —(CH₂)_{f}—N(R₁₁)—(R₁₀);
wherein said alkoxy, alkenyloxy, aryloxy, heteroaryl, alkylsulfonyl, S-alkyl, heterocycloalkyl, heterocycloalkylamino, heterocycloalkylaminoalkyl, heterocycloalkylalkyl alkoxyalkylaminoalkyl, heterocycloalkylalkylaminoalkyl, aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl, heteroaryl, heteroarylalkyl, alkylheteroaryl, heteroarylalkylamino, heteroarylalkylaminoalkyl, arylalkyloxy and arylsulfonyl groups are each optionally substituted with up to five independently selected R₆₁ groups; and said alkyl is optionally substituted with up to five independently selected R₆₀ groups;
f is 0, 1, 2, 3, 4, 5 or 6;
R₁₁ is H, alkyl or arylalkyl;
R₁₀ is alkyl, alkenyl, cycloalkyl, aryl, arylalkyl, heteroarylalkyl, or cycloalkylalkyl, wherein said arylalkyl, aryl and heteroaryl are each optionally substituted with are each optionally substituted with up to three independently selected R₆₁ groups; and said alkyl is optionally substituted with up to three independently selected R₆₀ groups;
or R₁₁ and R₁₀ together with the nitrogen to which they are attached can form a heterocyclic ring that is optionally substituted with up to three independently selected R₆₁ groups;
R₁₂ and R₁₃ are each independently H, alkyl or arylalkyl;
R₂₀ and R₂₁ are each independently H, alkyl or arylalkyl, wherein said arylalkyl is optionally substituted with up to three independently selected R₆₁ groups, and said alkyl is optionally substituted with up to three independently selected R₆₀ groups; and
R₅₀ is a group of Formula III: 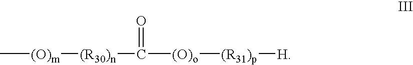
wherein m, n, o and p are each 0 or 1; and
R₃₀ and R₃₁ are each independently C₁₋₆alkyl.

42. A method for treating HCV in a patient suffering therefrom, comprising administering to said patient a therapeutically effective amount of a substituted oxoazepanylacetamide.

43. A method for treating HCV in a patient suffering therefrom, comprising administering to said patient a therapeutically effective amount of an oxoazepanylphenoxyacetamide.

44. A method for treating SARS in a patient suffering therefrom, comprising administering to said patient a therapeutically effective amount of a substituted oxoazepanylacetamide.

45. A method for treating SARS in a patient suffering therefrom, comprising administering to said patient a therapeutically effective amount of a substituted oxoazepanylphenoxyacetamide.

46. The method of claim 34 wherein said viral infection is HCV.

47. The method of claim 34 wherein said viral infection is SARS.
